(19) United States
(12) Patent Application Publication
(10) Pub. No.: US 2008/0194638 A1
(43) Pub. Date: Aug. 14, 2008

(54) BIOAVAILABLE FORMULATIONS OF HETEROCYCLIC COMPOUNDS

(75) Inventors: Mahendra G. DEDHIYA, Pomona, NY (US); Rahul Surana, Coram, NY (US); Suneel K. Rastogi, Ballwin, MI (US); Anil Chhetrry, Holtsville, NY (US)

Correspondence Address: Forest Laboratories, Inc.;Attn: Charles S. Ryan, 909 3rd Avenue, New York, NY, 10022, US

(73) Assignee: Forest Laboratories Holdings Limited, Hamilton (BM)

(21) Appl. No.: 12/028,058

(22) Filed: Feb. 8, 2008

Related U.S. Application Data
(60) Provisional application No. 60/889,009, filed on Feb. 9, 2007.
(60) Provisional application No. 60/896,353, filed on Mar. 22, 2007.

(51) Int. Cl. A61K 31/443; C07D 405/02; A61P 29/00; A61P 11/00; A61P 37/00

(52) U.S. Cl. 514/337; 546/284.1

(57) ABSTRACT

The present invention relates to bioavailable pharmaceutical formulations of heterocyclic compounds, such as such as N-(3,5-dichloropyrid-4-yl)-4-difluoromethoxy-8-methanesulfonamido-dibenzo[b,d]furan-1-carboxamide (oglemilast) and pharmaceutically acceptable salts thereof, to processes for their preparation and to methods of treatment using the same. The present invention also relates to substantially pure amorphous forms of heterocyclic compounds, such as oglemilast. The invention is particularly directed to bioavailable pharmaceutical oral dosage forms containing amorphous oglemilast.

This application claims the benefit of U.S. Application Ser. No. 60/889,009, filed Feb. 9, 2007 and U.S. Application Ser. No. 60/896,353, filed Mar. 22, 2007, the entire disclosures of each of which are hereby incorporated by reference.

### FIELD OF THE INVENTION

The present invention relates to bioavailable pharmaceutical formulations of heterocyclic compounds, such as such as N-(3,5-dichloropyrid-4-yl)-4-difluoromethoxy-8-methanesulfonamido-dibenzo[b,d]furan-1-carboxamide (oglemilast) and pharmaceutically acceptable salts thereof, to processes for their preparation and to methods of treatment using the same. The present invention also relates to substantially pure amorphous forms of heterocyclic compounds, such as oglemilast. The invention is particularly directed to bioavailable pharmaceutical oral dosage forms containing amorphous oglemilast.

### BACKGROUND OF THE INVENTION

Hormones are compounds that variously affect cellular activity. In many respects, hormones act as messengers to trigger specific cellular responses and activities. Many effects produced by hormones, however, are not caused by the singular effect of just the hormone. Instead, the hormone first binds to a receptor, thereby triggering the release of a second compound that goes on to affect the cellular activity. In this scenario, the hormone is known as the first messenger while the second compound is called the second messenger. Cyclic adenosine monophosphate (adenosine 3′,5′-cyclic monophosphate, “cAMP” or “cyclic AMP”) is known as a second messenger for hormones including epinephrine, glucagon, calcitonin, corticotrophin, lipotropin, luteinizing hormone, norepinephrine, parathyioid hormone, thyroid-stimulating hormone, and vasopressin. Thus, cAMP mediates cellular responses to hormones. Cyclic AMP also mediates cellular responses to various neurotransmitters.

Phosphodiesterases (“PDE”) are a family of enzymes that metabolize 3′,5′ cyclic nucleotides to 5′ nucleoside monophosphates, thereby terminating cAMP second messenger activity. A particular phosphodiesterase, phosphodiesterase-4 (“PDE4”, also known as “PDE-IV”), which is a high affinity, cAMP specific, type IV PDE, has generated interest as potential targets for the development of novel anti-asthmatic and anti-inflammatory compounds. The PDE4 enzyme family consists of four genes, which produce 4 isoforms of the PDE4 enzyme designated PDE4A, PDE4B, PDE4C, and PDE4D [See, e.g., Wang et al., Biochem. Biophys. Res. Comm., 234, 320-324 (1997)]. In addition, various splice variants of each PDE4 isoform have been identified.

Each of the four known PDE4 gene products is believed to play varying roles in allergic and/or inflammatory responses. Thus, it is believed that inhibition of PDE4, particularly the specific PDE4 isoforms that produce detrimental responses, can beneficially affect allergy and inflammation symptoms.

U.S. Patent Publication No. 2005/0027129 discloses heterocyclic compounds useful as PDE IV inhibitors for the treatment of, for example, inflammation and allergic disorders. These compounds are of the general formula:

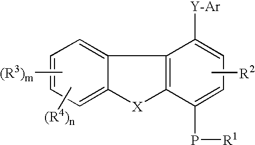

wherein R¹-R⁴, P, X, Y, m, n and Ar are as defined therein. One such compound is N-(3,5-dichloropyrid-4-yl)-4-difluoromethoxy-8-methanesulfonamido-dibenzo[b,d]furan-1-carboxamide, the International nonproprietary name for which is oglemilast. The pharmacology and safety profiles for oglemilast have been described in, for example, Eur. Respir. J. (2004) 24 (Suppl 48): Abst 1391. Both US 2005/0027129 and International Publication No. WO 2006/040652 disclose methods for preparing oglemilast and pharmaceutically acceptable salts thereof, such as the sodium salt (see, e.g., Examples 30 and 31 of US publication No. 2005/0027129) and generically disclose formulations of oglemilast and the corresponding sodium salt. In these formulations, the active ingredient is present substantially in a crystalline form. These conventional formulations, however, suffer from low bioavailability, because the solubility of the crystalline active ingredients is low. For example, crystalline oglemilast has a solubility of approximately 0.2 μg/mL. In an attempt to increase the bioavailability of formulations containing, e.g., oglemilast, salt forms of the active ingredient have typically been used. Although the solubility of the crystalline sodium salt of oglemilast is somewhat higher, at approximately 140 μg/mL, the bioavailability of a formulation substantially containing crystalline oglemilast sodium is still limited.

Therefore, there remains a need in the art to provide formulations, e.g., oral dosage forms, containing heterocyclic compounds, such as oglemilast, and pharmaceutically acceptable salts thereof, in which the formulations exhibit improved bioavailability.

### SUMMARY OF THE INVENTION

The present invention relates to bioavailable pharmaceutical formulations of heterocyclic compounds, such as such as N-(3,5-dichloropyrid-4-yl)-4-difluoromethoxy-8-methanesulfonamido-dibenzo[b,d]furan-1-carboxamide (oglemilast) and pharmaceutically acceptable salts thereof, to processes for their preparation and to methods of treatment using the same. The present invention also relates to substantially pure amorphous forms of heterocyclic compounds, such as oglemilast. The invention is particularly directed to bioavailable pharmaceutical oral dosage forms containing amorphous oglemilast.

### BRIEF DESCRIPTION OF THE DRAWINGS

FIGS. 1a and 1b show powder X-Ray diffraction spectra for crystalline oglemilast sodium salt, and crystalline and amorphous forms of oglemilast.

FIG. 2 shows the mean in vivo plasma concentrations for Formulations I-IV of Example 1 when administered to humans at a dose of 12 mg.

FIG. 3 shows the mean plasma concentrations in dogs for (i) a tablet formulation of the present invention, (ii) a solution formulation of the present invention (iii) a conventional solution formulation, and (iv) a conventional dry powder suspension.

FIG. 4 shows the pharmacokinetic profile for formulations G and H of Example 6.

FIG. 5 shows the pharmacokinetic profile for formulations J and K of Example 7.

FIG. 6 shows a granulation process for preparing oglemilast granules.

FIG. 7 shows a blending and compression process for preparing oglemilast tablets.

FIG. 8 shows a linear regression of mean steady state area under the curve (AUC₀₋₂₄) versus dose values following in vivo administration of oglemilast tablets at doses of 0.1 mg, 0.6 mg, 1.25 mg and 2.5 mg per day.

FIG. 9 shows a linear regression of mean steady state peak plasma concentration (Cₘₐₓ) versus dose values following in vivo administration of oglemilast tablets at doses of 0.1 mg, 0.6 mg, 1.25 mg and 2.5 mg per day.

### DETAILED DESCRIPTION OF THE INVENTION

The present invention relates to formulations of heterocyclic compounds suitable for oral administration, wherein the heterocyclic compounds are bioavailable. The formulations of the present invention provide improved dissolution profiles and increased bioavailability of the active ingredient when compared to conventional formulations.

Applicants have discovered that crystalline salts of various heterocyclic compounds, such as salts of the compound oglemilast (e.g., crystalline oglemilast sodium) readily convert to low solubility, and hence low bioavailability, crystalline non-salt forms of the heterocyclic compound (e.g., crystalline oglemilast) upon exposure to aqueous media. Without wishing to be bound by theory, applicants believe that the conversion of the crystalline salt form to the crystalline non-salt form of the heterocyclic compound occurs in two stages: the collapse of the crystalline salt lattice resulting in a high bioavailability amorphous intermediate, followed by crystallization to generate the low bioavailability crystalline non-salt form. In the presence of aqueous media, both steps are fast, resulting in rapid conversion.

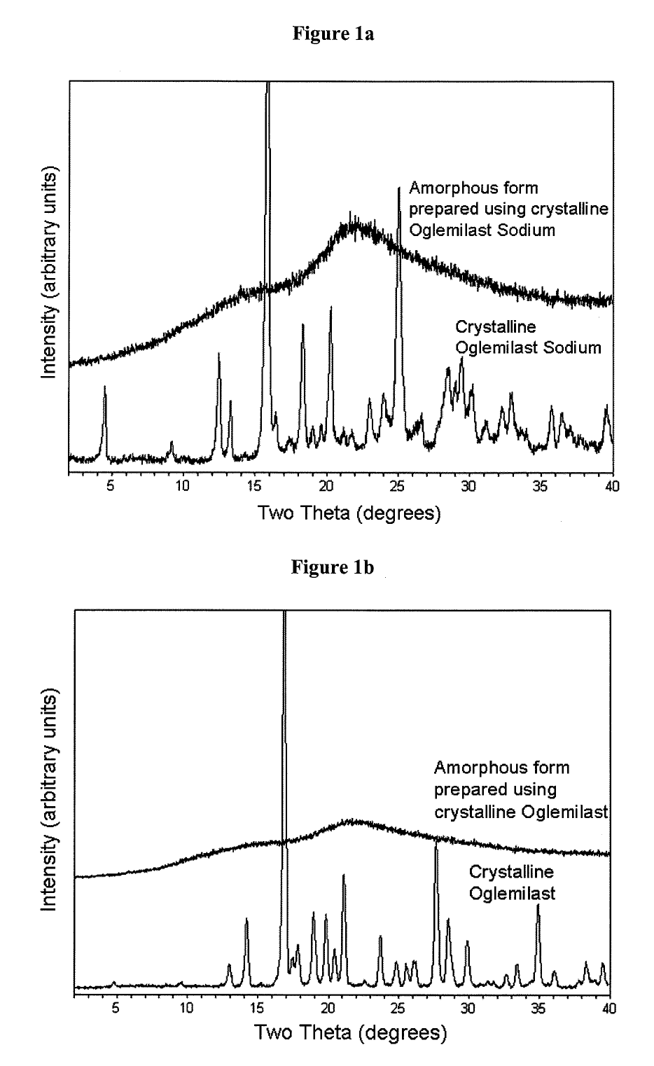

The crystalline and amorphous forms of oglemilast and the crystalline form of oglemilast sodium salt can readily be distinguished by powder X-Ray Diffraction (XRD). See FIGS. 1a and 1b.

Applicants have surprisingly found that the conversion of amorphous oglemilast to crystalline oglemilast can be retarded by the addition of certain excipients, thereby allowing for the preparation of high bioavailability formulations containing amorphous oglemilast. In the formulations of the present invention, the oglemilast is stabilized in an amorphous (and thus highly bioavailable) form and does not convert to low bioavailability crystalline oglemilast.

Accordingly, applicants have developed formulations containing heterocyclic compounds, such as oglemilast, and pharmaceutically acceptable salts thereof, in which the amount of crystalline heterocyclic compound is minimized. In the formulations of the present invention, the active ingredient remains in soluble form in the gastrointestinal (GI) tract, thereby resulting in higher bioavailability of the active ingredient(s). Formulations having higher bioavailability are desirable as they allow patient dosing at lower levels. Moreover, bioavailable formulations containing such heterocyclic compounds as the active ingredient may now be prepared by converting the oglemilast to a higher bioavailability form

Further, applicants have developed bioavailable liquid formulations (e.g. solutions) containing heterocyclic compounds such as oglemilast. In these liquid formulations, the precipitation of the crystalline form of the active ingredient is minimized, and hence the bioavailability of the liquid formulation is enhanced.

In one aspect, the present invention is directed to a substantially pure amorphous compound of formula (I):

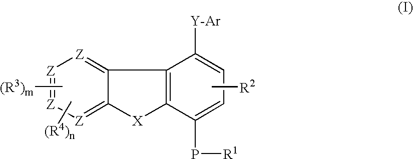

wherein

R¹, R² and R³ are each independently selected from the group consisting of hydrogen, substituted or unsubstituted alkyl, substituted or unsubstituted alkenyl, substituted or unsubstituted alkynyl, substituted or unsubstituted cycloalkyl, substituted or unsubstituted cycloalkylakyl, substituted or unsubstituted cycloalkenyl, substituted or unsubstituted aryl, substituted or unsubstituted arylalkyl, substituted or unsubstituted heteroaryl, substituted or unsubstituted heterocyclic group, substituted or unsubstituted heterocyclylalkyl, substituted or unsubstituted heteroarylalkyl, nitro, —OH, cyano, formyl, acetyl, halogen, protecting groups, —C(O)R^{a}, —C(O)OR^{a}, —C(O)NR^{a}R^{a}, —S(O)qR^{a}, S(O)_{q}NR^{a}R^{a}, —NRR^{a}, —OR^{a}, and —SR^{a},

or two R³ substituents ortho to each other, may be joined to a form a saturated or unsaturated 3-7 membered cyclic ring which may optionally include up to two heteroatoms which may be same or different selected from O, NR^{a} and S;

R⁴ is —NR⁵R⁶; wherein R⁵ and R⁶ are each independently selected from the group consisting of hydrogen, substituted or unsubstituted alkyl, substituted or unsubstituted alkenyl, substituted or unsubstituted alkynyl, substituted or unsubstituted cycloalkyl, substituted or unsubstituted cycloalkylakyl, substituted or unsubstituted cycloalkenyl, substituted or unsubstituted aryl, substituted or unsubstituted arylalkyl, substituted or unsubstituted heteroaryl, substituted or unsubstituted heterocyclic ring, substituted or unsubstituted heterocyclylalkyl, substituted or unsubstituted heteroarylalkyl, nitro, —OH, cyano, halogen, —C(O)R^{a}, —C(O)OR^{a}, —C(O)NRaR^{a}, —S(O)_{q}R^{a}, —S(O)_{q}NR^{a}R^{a}, —C(═NR^{a})R^{a}, —C(═NR^{a})NR^{a}R^{a}, —C(═S)NR^{a}R^{a}, —C(═S)R^{a}, —N═C(R^{a}R^{a})—, —NR^{a}R^{a}, —OR^{a}, —SR^{a}, and protecting groups,

or R⁵ and R⁶ may be joined to a form a saturated or unsaturated 3-7 membered cyclic ring, which may optionally include up to two heteroatoms which may be same or different selected from O, NR^{a} and S;

Ar is selected from the group consisting of substituted or unsubstituted aryl, substituted or unsubstituted arylalkyl, substituted or unsubstituted heterocyclic ring and substituted or unsubstituted heteroaryl ring;

X is selected from the group consisting of O, S(O)_{q} and NR^{a};

Y is selected from the group consisting of —C(O)NR⁷, —NR⁷S(O)_{q}, —S(O)_{q}NR⁷ and —NR⁷C(O);

each Z is independently C or N;

R⁷ is selected from the group consisting of hydrogen, substituted or unsubstituted alkyl, hydroxyl, —OR^{a}, substituted or unsubstituted aryl, and substituted or unsubstituted heterocyclic ring;

p is chosen from O and S;

m represents 0-3; n represents 1-4; q represents 0, 1 or 2;

and

R^{a} is selected from the group consisting of hydrogen, substituted or unsubstituted alkyl, substituted or unsubstituted alkenyl, substituted or unsubstituted alkynyl, substituted or unsubstituted cycloalkyl, substituted or unsubstituted cycloalkylakyl, substituted or unsubstituted cycloalkenyl, substituted or unsubstituted aryl, substituted or unsubstituted arylalkyl, substituted or unsubstituted heteroaryl, substituted or unsubstituted heterocyclic ring, substituted or unsubstituted heterocyclylalkyl, substituted or unsubstituted heteroarylalkyl, nitro, —OH, cyano, formyl, acetyl, halogen, protecting groups, —C(O)Ra, —C(O)OR^{a}, —C(O)NR^{a}R^{a}, —S(O)_{q}R^{a}, —S(O)_{q}NR^{a}R^{a}, —N^{a}R^{a}, —OR^{a} and —SR^{a}.

In one embodiment, R⁴ is not NH₂.

In additional embodiments, about 10% or more, about 20% or more, about 30% or more, about 35% or more, about 40% or more, about 45% or more, about 50% or more, about 55% or more, about 60% or more, about 65% or more, about 70% or more, about 75% or more, about 80% or more, about 85% or more, about 90% or more, about 95% or more, about 97.5% or more, about 98% or more, about 99% or more, or about 99.5% or more of the compound of Formula (I) is present in amorphous form. For example, about 20% or more, 40% or more, about 60% or more, about 80% or more or about 90% or more of the compound of Formula (I) is in amorphous form.

In an exemplary embodiment, the compound of formula I is oglemilast.

In another aspect, the present invention provides formulations that comprise, e.g., from about 0.05 mg to about 50 mg of a bioavailable form of a heterocyclic compound by retardation of precipitation of active drug.

In another aspect, the present invention provides formulations that comprise, e.g., from about 0.05 mg to about 50 mg of a containing/comprising amorphous form of a heterocyclic compound.

In one embodiment, the present invention relates to formulations containing about 10% or more of an amorphous compound of formula (I):

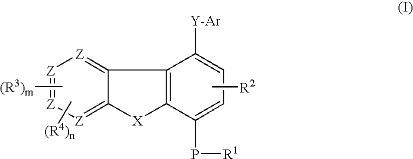

wherein

R¹, R² and R³ are each independently selected from the group consisting of hydrogen, substituted or unsubstituted alkyl, substituted or unsubstituted alkenyl, substituted or unsubstituted alkynyl, substituted or unsubstituted cycloalkyl, substituted or unsubstituted cycloalkylaryl, substituted or unsubstituted cycloalkenyl, substituted or unsubstituted aryl, substituted or unsubstituted arylalkyl, substituted or unsubstituted heteroaryl, substituted or unsubstituted heterocyclic group, substituted or unsubstituted heterocyclylalkyl, substituted or unsubstituted heteroarylalkyl, nitro, —OH, cyano, formyl, acetyl, halogen, protecting groups, —C(O)R^{a}, —C(O)OR^{a}, —C(O)NR^{a}R^{a}, —S(O)qR^{a}, S(O)_{q}NR^{a}R^{a}, —NRR^{a}, —OR^{a}, and —SR^{a},

or two R³ substituents ortho to each other, may be joined to a form a saturated or unsaturated 3-7 membered cyclic ring which may optionally include up to two heteroatoms which may be same or different selected from O, NR^{a} and S;

R⁴ is —NR⁵R⁶; wherein R⁵ and R⁶ are each independently selected from the group consisting of hydrogen, substituted or unsubstituted alkyl, substituted or unsubstituted alkenyl, substituted or unsubstituted alkynyl, substituted or unsubstituted cycloalkyl, substituted or unsubstituted cycloalkylaryl, substituted or unsubstituted cycloalkenyl, substituted or unsubstituted aryl, substituted or unsubstituted arylalkyl, substituted or unsubstituted heteroaryl, substituted or unsubstituted heterocyclic ring, substituted or unsubstituted heterocyclylalkyl, substituted or unsubstituted heteroarylalkyl, nitro, —OH, cyano, halogen, —C(O)R^{a}, —C(O)OR^{a}, —C(O)NRaR^{a}, —S(O)_{q}R^{a}, —S(O)_{q}NR^{a}R^{a}, —C(═NR^{a})R^{a}, —C(═NR^{a})NR^{a}R^{a}, —C(═S)NR^{a}R^{a}, —C(═S)R^{a}, —N═C(R^{a}R^{a})—, —NR^{a}R^{a}, —OR^{a}, —SR^{a}, and protecting groups,

or R⁵ and R⁶ may be joined to a form a saturated or unsaturated 3-7 membered cyclic ring, which may optionally include up to two heteroatoms which may be same or different selected from O, NR^{a} and S;

Ar is selected from the group consisting of substituted or unsubstituted aryl, substituted or unsubstituted arylalkyl, substituted or unsubstituted heterocyclic ring and substituted or unsubstituted heteroaryl ring;

X is selected from the group consisting of O, S(O)_{q} and NR^{a};

Y is selected from the group consisting of —C(O)NR⁷, —NR⁷S(O)_{q}, —S(O)_{q}NR⁷ and —NR⁷C(O);

each Z is independently C or N;

R⁷ is selected from the group consisting of hydrogen, substituted or unsubstituted alkyl, hydroxyl, —OR^{a}, substituted or unsubstituted aryl, and substituted or unsubstituted heterocyclic ring;

p is chosen from O and S;

m represents 0-3; n represents 1-4; q represents 0, 1 or 2;

and

R^{a} is selected from the group consisting of hydrogen, substituted or unsubstituted alkyl, substituted or unsubstituted alkenyl, substituted or unsubstituted alkynyl, substituted or unsubstituted cycloalkyl, substituted or unsubstituted cycloalkylakyl, substituted or unsubstituted cycloalkenyl, substituted or unsubstituted aryl, substituted or unsubstituted arylalkyl, substituted or unsubstituted heteroaryl, substituted or unsubstituted heterocyclic ring, substituted or unsubstituted heterocyclylalkyl, substituted or unsubstituted heteroarylalkyl, nitro, —OH, cyano, formyl, acetyl, halogen, protecting groups, —C(O)Ra, —C(O)OR^{a}, —C(O)NR^{a}R^{a}, —S(O)_{q}R^{a}, —S(O)_{q}NR^{a}R^{a}, —N^{a}R^{a}, —OR^{a} and —SR^{a}.

In one embodiment, R⁴ is not NH₂.

In additional embodiments, the present invention provides formulations that include a compound of formula I, wherein about 10% or more, about 20% or more, about 30% or more, about 35% or more, about 40% or more, about 45% or more, about 50% or more, about 55% or more, about 60% or more, about 65% or more, about 70% or more, about 75% or more, about 80% or more, about 85% or more, about 90% or more, about 95% or more, about 97.5% or more, about 98% or more, about 99% or more, or about 99.5% or more of the compound of formula (I) is present in amorphous form. For example, about 20% or more, about 40% or more, about 60% or more, about 80% or more or about 90% or more of the compound of formula (I) is present in amorphous form.

In additional embodiment, the present invention provides formulations that comprise, highly a bioavailable form of a heterocyclic compound by retardation of precipitation of active drug.

Without wishing to be bound by theory, Applicants believe that the compounds of formula I in the formulations of the present invention may be stabilized in an amorphous form by the presence of one or more excipients (e.g., PVP and HPMC) due to (i) dispersive interactions between the ring of the one or more excipients and the ring structure of formula I, and/or (ii) hydrogen bonding interactions between the one or more excipients and the substituents on the ring structure of formula I. Due to such dispersive and hydrogen bonding interactions, applicants believe that a stacking of the planar ring structure of formula I and excipient molecules occurs, inhibiting molecular motion and thus retarding crystallization of the non-salt form of the active ingredient. In addition, the presence of one or more excipients (e.g., PVP and HPMC) retards precipitation of active drug and thereby increases bioavailability. In exemplary embodiments, the formulations comprise oglemilast. In additional embodiments, the formulations further comprise oglemilast sodium.

In a further aspect, the present invention relates to a formulation comprising from about 0.05 mg to about 2.5 mg oglemilast, or a pharmaceutically acceptable salt thereof, wherein the single dose administration of formulation provides an in vivo plasma profile comprising (i) a mean Cₘₐₓ of more than about 2.1 ng/mL, (ii) a mean AUC₀₋₂₄ of more than about 26.3 ng hr/mL and (iii) a mean Tₘₐₓ of about 0.5 or more hours. For example, the formulation provides an in vivo plasma profile comprising (i) a mean Cₘₐₓ of more than about 2.3 ng/mL, (ii) a mean AUC₀₋₂₄ of more than about 28.9 ng.hr/mL and (iii) a mean Tₘₐₓ of about 0.8 or more hours. For further example, the formulation provides an in vivo plasma profile comprising (i) a mean Cₘₐₓ of more than about 2.5 ng/mL, (ii) a mean AUC₀₋₂₄ of more than about 36 ng.hr/mL and (iii) a mean Tₘₐₓ of about 1 or more hours.

In one embodiment, the formulation comprises about 0.1 mg oglemilast, or a pharmaceutically acceptable salt thereof, and the single dose administration of formulation provides an in vivo plasma profile comprising (i) a mean Cₘₐₓ of more than about 4.2 ng/mL, (ii) a mean AUC₀₋₂₄ of more than about 53 ng.hr/mL and (iii) a mean Tₘₐₓ of about 0.5 or more hours. For example, the formulation provides an in vivo plasma profile comprising (i) a mean Cₘₐₓ of more than about 4.6 ng/mL, (ii) a mean AUC₀₋₂₄ of more than about 58 ng.hr/mL and (iii) a mean Tₘₐₓ of about 0.8 or more hours. For further example, the formulation provides an in vivo plasma profile comprising (i) a mean Cₘₐₓ of more than about 5 ng/mL, (ii) a mean AUC₀₋₂₄ of more than about 63 ng.hr/mL and (iii) a mean Tₘₐₓ of about 1 or more hours.

In one embodiment, the formulation comprises about 0.2 mg oglemilast, or a pharmaceutically acceptable salt thereof, and the single dose administration of formulation provides an in vivo plasma profile comprising (i) a mean Cₘₐₓ of more than about 9.6 ng/mL, (ii) a mean AUC₀₋₂₄ of more than about 110 ng.hr/mL and (iii) a mean Tₘₐₓ of about 0.5 or more hours. For example, the formulation provides an in vivo plasma profile comprising (i) a mean Cₘₐₓ of more than about 10.5 ng/mL, (ii) a mean AUC₀₋₂₄ of more than about 121 ng.hr/mL and (iii) a mean Tₘₐₓ of about 0.8 or more hours. For further example, the formulation provides an in vivo plasma profile comprising (i) a mean Cₘₐₓ of more than about 11.5 ng/mL, (ii) a mean AUC₀₋₂₄ of more than about 132 ng.hr/mL and (iii) a mean Tₘₐₓ of about 1 or more hours.

In one embodiment, the formulation comprises about 0.4 mg oglemilast, or a pharmaceutically acceptable salt thereof, and the single dose administration of formulation provides an in vivo plasma profile comprising (i) a mean Cₘₐₓ of more than about 19.1 ng/mL, (ii) a mean AUC₀₋₂₄ of more than about 220 ng.hr/mL and (iii) a mean Tₘₐₓ of about 0.5 or more hours. For example, the formulation provides an in vivo plasma profile comprising (i) a mean Cₘₐₓ of more than about 21 ng/mL, (ii) a mean AUC₀₋₂₄ of more than about 242 ng.hr/mL and (iii) a mean Tₘₐₓ of about 0.8 or more hours. For further example, the formulation provides an in vivo plasma profile comprising (i) a mean Cₘₐₓ of more than about 23 ng/mL, (ii) a mean AUC₀₋₂₄ of more than about 264 ng.hr/mL and (iii) a mean Tₘₐₓ of about 1 or more hours.

In another embodiment, the formulation comprises about 0.6 mg oglemilast, or a pharmaceutically acceptable salt thereof, and the single dose administration of formulation provides an in vivo plasma profile comprising (i) a mean Cₘₐₓ of more than about 26 ng/mL, (ii) a mean AUC₀₋₂₄ of more than about 294 ng.hr/mL and (iii) a mean Tₘₐₓ of about 0.5 or more hours. For example, the formulation provides an in vivo plasma profile comprising (i) a mean Cₘₐₓ of more than about 28.5 ng/mL, (ii) a mean AUC₀₋₂₄ of more than about 323 ng.hr/mL and (iii) a mean Tₘₐₓ of about 0.8 or more hours. For further example, the formulation provides an in vivo plasma profile comprising (i) a mean Cₘₐₓ of more than about 31 ng/mL, (ii) a mean AUC₀₋₂₄ of more than about 353 ng.hr/mL and (iii) a mean Tₘₐₓ of about 1 or more hours.

In another embodiment, the formulation comprises about 0.8 mg oglemilast, or a pharmaceutically acceptable salt thereof, and the single dose administration of formulation provides an in vivo plasma profile comprising (i) a mean Cₘₐₓ of more than about 38 ng/mL, (ii) a mean AUC₀₋₂₄ of more than about 440 ng.hr/mL and (iii) a mean Tₘₐₓ of about 0.5 or more hours. For example, the formulation provides an in vivo plasma profile comprising (i) a mean Cₘₐₓ of more than about 42 ng/mL, (ii) a mean AUC₀₋₂₄ of more than about 484 ng.hr/mL and (iii) a mean Tₘₐₓ of about 0.8 or more hours. For further example, the formulation provides an in vivo plasma profile comprising (i) a mean Cₘₐₓ of more than about 46 ng/mL, (ii) a mean AUC₀₋₂₄ of more than about 528 ng.hr/mL and (iii) a mean Tₘₐₓ of about 1 or more hours

In a further embodiment, the formulation comprises about 1.25 mg oglemilast, or a pharmaceutically acceptable salt thereof, and the single dose administration of formulation provides an in vivo plasma profile comprising (i) a mean Cₘₐₓ of more than about 54 ng/mL, (ii) a mean AUC₀O₂₄ of more than about 631 ng.hr/mL and (iii) a mean Tₘₐₓ of about 0.5 or more hour. For example, the formulation provides an in vivo plasma profile comprising (i) a mean Cₘₐₓ of more than about 59 ng/mL, (ii) a mean AUC₀₋₂₄ of more than about 694 ng.hr/mL and (iii) a mean Tₘₐₓ of about 0.8 or more hours. For further example, the formulation provides an in vivo plasma profile comprising (i) a mean Cₘₐₓ of more than about 65 ng/mL, (ii) a mean AUC₀₋₂₄ of more than about 757 ng.hr/mL and (iii) a mean Tₘₐₓ of about 1 or more hours.

In yet another embodiment, the formulation comprises about 2.5 mg oglemilast, or a pharmaceutically acceptable salt thereof, and the single dose administration of formulation provides an in vivo plasma profile comprising (i) a mean Cₘₐₓ of more than about 129 ng/mL, (ii) a mean AUC₀₋₂₄ of more than about 1471 ng.hr/mL and (iii) a mean Tₘₐₓ of about 0.5 or more hours. For example, the formulation provides an in vivo plasma profile comprising (i) a mean Cₘₐₓ of more than about 142 ng/mL, (ii) a mean AUC₀O₂₄ of more than about 1618 ng.hr/mL and (iii) a mean Tₘₐₓ of about 0.8 or more hours. For further example, the formulation provides an in vivo plasma profile comprising (i) a mean Cₘₐₓ of more than about 154 ng/mL, (ii) a mean AUC₀₋₂₄ of more than about 1765 ng.hr/mL and (iii) a mean Tₘₐₓ of about 1 or more hours.

In yet another embodiment, the formulation comprises about 12 mg oglemilast, or a pharmaceutically acceptable salt thereof, and the single dose administration of formulation provides an in vivo plasma profile comprising (i) a mean Cₘₐₓ of more than about 264 ng/mL, (ii) a mean AUC₀₋₂₄ of more than about 4108 ng.hr/mL and (iii) a mean Tₘₐₓ of about 0.5 or more hours. For example, the formulation provides an in vivo plasma profile comprising (i) a mean Cₘₐₓ of more than about 291 ng/mL, (ii) a mean AUC₀₋₂₄ of more than about 4513 ng.hr/mL and (iii) a mean Tₘₐₓ of about 0.8 or more hours. For further example, the formulation provides an in vivo plasma profile comprising (i) a mean Cₘₐₓ of more than about 318 ng/mL, (ii) a mean AUC₀₋₂₄ of more than about 4920 ng.hr/mL and (iii) a mean Tₘₐₓ of about 1 or more hours.

In additional embodiments, the formulations include about 10% or more, about 20% or more, about 30% or more, about 35% or more, about 40% or more, about 45% or more, about 50% or more, about 55% or more, about 60% or more, about 65% or more, about 70% or more, about 75% or more, about 80% or more, about 85% or more, about 90% or more, about 95% or more, about 97.5% or more, about 98% or more, about 99% or more, or about 99.5% or more of amorphous oglemilast. For example, the formulations include about 20% or more, about 40% or more, about 60% or more, about 75% or more, about 80% or more or about 90% or more of amorphous oglemilast.

In another aspect, the present invention provides formulations that comprise of a bioavailable form of a heterocyclic compound due to retardation of precipitation of active drug.

In other embodiments, the present invention provides formulations, e.g. oral dosage forms, comprising about 20% or more amorphous oglemilast which provide an in vivo plasma profile comprising (i) a mean Cₘₐₓ of more than about 2.1 ng/mL, (ii) a mean AUC_{0-∞} of less than about 15,000 ng h/ml and (iii) a mean Tₘₐₓ of more than about 0.25 hour.

In other embodiments, the present invention provides formulations, e.g. oral dosage forms, comprising highly soluble form oglemilast which provide an in vivo plasma profile comprising (i) a mean Cₘₐₓ of more than about 2.1 ng/mL, (ii) a mean AUC_{0-∞} of less than about 15,000 ng h/ml and (iii) a mean Tₘₐₓ of more than about 0.25 hour.

In further embodiments, the formulations include about 0.8 mg oglemilast wherein about 20% or more of the oglemilast is amorphous, wherein the formulation provides an in vivo plasma profile comprising (i) a mean Cₘₐₓ of more than about 38 ng/mL, (ii) a mean AUC₀₋₁₂ of more than about 440 ng h/mL, and (iii) a mean Tₘₐₓ of more than about 0.25 hours.

In a further embodiment, the formulation provides an in vivo plasma profile comprising a mean Cₘₐₓ of more than about 2 ng/mL.

In a further aspect, the formulations of the present invention exhibit a dissolution rate of the active ingredient of about 50% or more in about 60 minutes or less. In another embodiment, the formulations exhibit a dissolution rate of the active ingredient of about 70% or more in about 60 minutes or less. In yet a further embodiment, the formulations exhibit a dissolution rate of the active ingredient of about 80% or more in about 60 minutes or less.

In a further aspect, the present invention relates to formulations, e.g., oral dosage forms, e.g., solutions and suspensions, that include (i) about 20% or more of solubilized compound of formula (I) (e.g., oglemilast) and (ii) one or more excipients, wherein the one or more excipients are present in an amount sufficient to retard formation of crystalline oglemilast upon exposure to an aqueous media.

Suitable excipients that may be used to retard formation of crystalline compound of formula (I) upon exposure to an aqueous media, include, but are not limited to, povidone (PVP), polyethylene glycol (PEG), celluloses (e.g., hydroxypropyl methylcellulose (HPMC), hydroxypropyl cellulose (HPC)), pregelatinized starch, povidone-vinyl acetate (PVP-VA) copolymers, cyclodextrins (e.g., hydroxypropyl beta cyclodextrin), disaccharides (e.g., sucrose, trehalose) polysaccharides (e.g., dextran) and combinations thereof. In one embodiment the excipient is povidone (PVP). In another embodiment the excipient is hydroxypropyl methylcellulose (HPMC).

In certain embodiments, the ratio of active ingredient to the one or more excipients used to retard formation of crystalline oglemilast upon exposure to an aqueous media is from about 1:0.05 w/w to about 1:50 w/w.

In one embodiment, the active ingredient in the formulation has a particle size distribution characterized by an X₉₀ of less than about 10 μm.

Applicants have also discovered that conventional liquid formulations containing heterocyclic compounds such as oglemilast and salts thereof exhibit lower bioavailability than would generally be expected for a solution formulation. This is unexpectedly due to the precipitation of the crystalline form of the active ingredient, oglemilast (formula I). However, the use of the one or more excipients described above also allows the stabilization of a solubilized form of the active ingredient thereby allowing bioavailable liquid formulations to be prepared.

Accordingly, in a further aspect, the present invention provides liquid formulations, e.g. solutions and suspensions, that include solubilized form or of a compound of formula I (e.g., oglemilast) stabilized by one of more excipients.

In one embodiment, the formulation is a solution having a pH greater than about 7.

In further embodiments, the formulations of the present invention include about 0.05 mg, about 0.1 mg, about 0.2 mg, about 0.3 mg, about 0.4 mg, about 0.5 mg, about 0.6 mg, about 0.75 mg, about 0.8 mg, about 0.9 mg about 1 mg, about 1.25 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 10.5 mg, about 11 mg, about 11.5 mg, about 12 mg, about 12.5 mg, about 13 mg, about 13.5 mg, about 14 mg, about 14.5 mg, about 15 mg, about 15.5 mg, about 16 mg, about 16.5 mg, about 17 mg, about 17.5 mg, about 18 mg, about 18.5 mg, about 19 mg, about 19.5 mg, about 20 mg, about 20.5 mg, about 21 mg, about 21.5 mg, about 22 mg, about 22.5 mg, about 23 mg, about 23.5, about 24 mg, about 24.5 mg, about 25 mg, about 25.5 mg, about 26 mg, about 26.5 mg, about 27 mg, about 27.5 mg, about 28 mg, about 28.5 mg, about 29 mg, about 29.5 mg, or about 30 mg active ingredient. In yet further embodiments, the formulations include an amount of the active ingredient which ranges between any two of these dosage amounts (e.g., from about 0.05 to about 50 mg, from about 0.1 to about 3.0 mg, from about 0.1 to about 2 mg, from about 0.2 to about 1.25 mg). In further embodiments, the formulations include between about 0.1 mg and about 2 mg of active ingredient. For example, the formulations include about 0.1 mg, about 0.2, about 0.4 mg, about 0.6 mg, about 0.8 mg, about 0.9 mg, about 1.25 mg or about 2.5 mg of active ingredient (e.g., about 0.1 mg, about 0.2 mg, about 0.4 mg, about 0.6 mg, about 0.8 mg, about 0.9 mg about 1.25 mg or about 2.5 mg of active ingredient).

### Pharmaceutically Acceptable Salts

As stated above, one aspect of the present invention provides bioavailable formulations comprising amorphous active ingredients, wherein the need to prepare a salt form of the active ingredient in order to enhance bioavailability has been obviated. However, the formulations of the present invention may contain salt forms of the active ingredient. Suitable pharmaceutically acceptable salts include those obtained by reacting the main compound, functioning as a base with an inorganic or organic acid to form a salt, for example, salts of hydrochloric acid, sulfuric acid, phosphoric acid, methane sulfonic acid, camphor sulfonic acid, oxalic acid, maleic acid, succinic acid, citric acid, formic acid, hydrobromic acid, benzoic acid, tartaric acid, fumaric acid, salicylic acid, mandelic acid, and carbonic acid. Pharmaceutically acceptable salts also include those in which the main compound functions as an acid and is reacted with an appropriate base to form, e.g., sodium, potassium, calcium, magnesium, ammonium, and choline salts. Those skilled in the art will further recognize that acid addition salts of the claimed compounds may be prepared by reaction of the compounds with the appropriate inorganic or organic acid via any of a number of known methods. Alternatively, alkali and alkaline earth metal salts can be prepared by reacting the compounds of the invention with the appropriate base via a variety of known methods.

The following are further examples of acid salts that can be obtained by reaction with inorganic or organic acids: acetates, adipates, alginates, citrates, aspartates, benzoates, benzenesulfonates, bisulfates, butyrates, camphorates, digluconates, cyclopentanepropionates, dodecylsulfates, ethanesulfonates, glucoheptanoates, glycerophosphates, hemisulfates, heptanoates, hexanoates, fumarates, hydrobromides, hydroiodides, 2-hydroxy-ethanesulfonates, lactates, maleates, methanesulfonates, nicotinates, 2-naphthalenesulfonates, oxalates, palmoates, pectinates, persulfates, 3-phenylpropionates, picrates, pivalates, propionates, succinates, tartrates, thiocyanates, tosylates, mesylates and undecanoates.

In one embodiment, the pharmaceutically acceptable salt is a sodium, or potassium, or, magnesium, or calcium salt. For example, the pharmaceutically acceptable salt is a sodium salt.

### Compositions

In certain embodiments, the formulations of the present invention can be adapted for administration as, for example, pills, tablets, capsules, powders, caplets, troches, dry powder suspensions, wafers, lozenges, orally disintegrating films, orally disintegrating tablets, and modified release dosage forms, and the like.

In certain embodiments, the formulations of the present invention can be adapted for administration as, for example, aqueous and non-aqueous solutions, emulsions, suspensions, syrups, and elixirs. Such dosage forms can also contain suitable inert diluents known in the art such as water and suitable excipients known in the art such as preservatives, wetting agents, sweeteners, flavorants, as well as agents for emulsifying and/or suspending the compounds of the invention.

The formulations of the present invention may also include additional pharmaceutically acceptable carriers, diluents and excipients known in the art, including, but not limited to, suspending agents, solubilizers, buffering agents, binders, disintegrants, preservatives, colorants, flavorants, lubricants, solvents, glidants and the like. See, for example, the Handbook of Pharmaceutical Excipients, American Pharmaceutical Association (current edition); Pharmaceutical Dosage Forms: Tablets (Lieberman, Lachman and Schwartz, editors) current edition, published by Marcel Dekker, Inc., as well as Remington's Pharmaceutical Sciences (Arthur Osol, editor), 1553-1593 (current edition).

### Processes

The present invention also relates to processes for preparing formulations of the present invention

In one embodiment, the present invention relates to a process comprising (a) combining crystalline oglemilast, in the form of a pharmaceutically acceptable salt thereof, and one or more excipients in the presence of water wherein the one or more excipients is/are present in an amount sufficient to stabilize the intermediate amorphous oglemilast formed, and hence retard formation of crystalline oglemilast.

Granulation

In additional embodiments, formulations may be prepared wherein the process further comprises the steps of (b) granulating the mixture formed in step (a) with one or more substrates (c) drying the resulting product, and (d) blending the product from step (c) with one or more pharmaceutical acceptable excipients. In a further embodiment, the process further comprises (e) compressing the blend from step (d) into tablets.

In additional embodiments, step (b) is performed at a product temperature of between about 25° C. and about 60° C., for example, between about 27° C. and about 50° C. In another embodiment, the one or more substrates in step (b) are selected from celluloses (e.g., silicified microcrystalline cellulose) and starches (e.g., sodium starch glycolate) and mixtures thereof. Step (b) can be performed in a fluidized bed.

In further embodiments, step (c) is conducted at a product temperature of about 30° C. to about 50° C., for example at a temperature of about 40° C. to about 45° C.

In additional embodiments, the one or more pharmaceutical excipients in step (d) are selected from disintegrants (e.g., starches, such as sodium starch glycolate), diluents (e.g., celluloses such as silicified microcrystalline cellulose), glidants (e.g., colloidal silicon dioxide, talc), and lubricants (e.g., magnesium stearate), and the like.

Methods of Treatment

The compounds of formula I, such as oglemilast, and pharmaceutically acceptable salts thereof, such as oglemilast sodium salt, are phosphodiesterase 4 inhibitors. As a result, the formulations of the present invention are useful in the treatment of a variety of disease states characterized by decreased cyclic AMP levels and/or elevated phosphodiesterase 4 levels, for example allergic and inflammatory diseases and disorders.

Thus, in accordance with a further embodiment of the invention, there is provided a method of treating allergic and inflammatory disease states, comprising administering to a patient in need thereof an effective amount of a formulation of the present invention.

Such disease states include, but are not limited to, asthma, chronic bronchitis, chronic obstructive pulmonary disease (COPD), atopic dermatitis, urticaria, allergic rhinitis, allergic conjunctivitis, vernal conjunctivitis, esoniophilic granuloma, psoriasis, inflammatory arthritis, rheumatoid arthritis, septic shock, ulcerative colitis, Crohn's disease, reperfusion injury of the myocardium and brain, chronic glomerulonephritis, endotoxic shock, adult respiratory distress syndrome, cystic fibrosis, arterial restenosis, artherosclerosis, keratosis, rheumatoid spondylitis, osteoarthritis, pyresis, diabetes mellitus, pneumoconiosis, chronic obstructive airways disease, toxic and allergic contact eczema, atopic eczema, seborrheic eczema, lichen simplex, sunburn, pruritis in the anogenital area, alopecia areata, hypertrophic scars, discoid lupus erythematosus, systemic lupus erythematosus, follicular and wide-area pyodermias, endogenous and exogenous acne, acne rosacea, Beghet's disease, anaphylactoid purpura nephritis, inflammatory bowel disease, leukemia, multiple sclerosis, gastrointestinal diseases, autoimmune diseases, and the like.

Preferred inflammatory disorders include asthma, bronchial asthma, chronic obstructive pulmonary disease, allergic rhinitis, eosinophilic granuloma, nephritis, rheumatoid arthritis, cystic fibrosis, chronic bronchitis, multiple sclerosis, Crohn's disease, psoraisis, uticaria, adult vernal cojunctivitis, respiratory distress syndrome, rhematoid spondylitis, osteoarthritis, gouty arthritis, uteltis, allergic conjunctivitis, inflammatory bowel conditions, ulcerative colitis, eczema, atopic dermatitis and chronic inflammation. Further preferred are allergic inflammatory conditions.

Preferred inflammatory disorders include, but are not limited to, chronic obstructive pulmonary disease (COPD) and asthma.

Also preferred are inflammatory conditions and immune disorders selected from inflammatory conditions or immune disorders of the lungs, joints, eyes, bowels, skin and heart.

Additionally preferred are inflammatory conditions chosen from the group consisting of bronchial asthma, nephritis, and allergic rhinitis.

Another object of the invention is a method for abating inflammation in an affected organ or tissue including delivering to the organ or tissue a therapeutically effective amount of an oral dosage form of the present invention.

Another object of the invention is a method of treating diseases of the central nervous system in a subject in need thereof which comprises administering to said subject a therapeutically effective amount of an oral dosage form of the present invention

Preferred diseases of the central nervous system include, but are not limited to, depression, amnesia, dementia, Alzheimer's disease, cardiac failure, shock and cerebrovascular disease.

Another object of the invention is a method of treating insulin resistant diabetes in a subject in need thereof which comprises administering to said subject a therapeutically effective amount of an oral dosage form of the present invention

The four classic symptoms of acute inflammation are redness, elevated temperature, swelling, and pain in the affected area, and loss of function of the affected organ.

Symptoms and signs of inflammation associated with specific conditions include:

rheumatoid arthritis—pain, swelling, warmth and tenderness of the involved joints; generalized and morning stiffness;

insulin-dependent diabetes mellitus—insulitis; this condition can lead to a variety of complications with an inflammatory component, including: retinopathy, neuropathy, nephropathy, coronary artery disease, peripheral vascular disease, and cerebrovascular disease;

autoimmune thyroiditis—weakness, constipation, shortness of breath, puffiness of the face, hands and feet, peripheral edema, bradycardia;

multiple sclerosis—spasticity, blurry vision, vertigo, limb weakness, paresthesias;

uveoretinitis—decreased night vision, loss of peripheral vision;

lupus erythematosus—joint pain, rash, photosensitivity, fever, muscle pain, puffiness of the hands and feet, abnormal urinalysis (hematuria, cylinduria, proteinuria), glomerulonephritis, cognitive dysfunction, vessel thrombosis, pericarditis;

scleroderma—Raynaud's disease; swelling of the hands, arms, legs and face; skin thickening; pain, swelling and stiffness of the fingers and knees, gastrointestinal dysfunction, restrictive lung disease; pericarditis; renal failure;

other arthritic conditions having an inflammatory component such as rheumatoid spondylitis, osteoarthritis, septic arthritis and polyarthritis—fever, pain, swelling, tenderness;

other inflammatory brain disorders, such as meningitis, Alzheimer's disease, AIDS dementia encephalitis—photophobia, cognitive dysfunction, memory loss;

other inflammatory eye inflammations, such as retinitis—decreased visual acuity;

inflammatory skin disorders, such as, eczema, other dermatitis (e.g., atopic, contact), psoriasis, burns induced by UV radiation (sun rays and similar UV sources)—erythema, pain, scaling, swelling, tenderness;

inflammatory bowel disease, such as Crohn's disease, ulcerative colitis—pain, diarrhea, constipation, rectal bleeding, fever, arthritis;

asthma—shortness of breath, wheezing;

other allergy disorders, such as allergic rhinitis—sneezing, itching, runny nose conditions associated with acute trauma such as cerebral injury following stroke—sensory loss, motor loss, cognitive loss;

heart tissue injury due to myocardial ischemia—pain, shortness of breath;

lung injury such as that which occurs in adult respiratory distress syndrome—shortness of breath, hyperventilation, decreased oxygenation, pulmonary infiltrates;

inflammation accompanying infection, such as sepsis, septic shock, toxic shock syndrome—fever, respiratory failure, tachycardia, hypotension, leukocytosis;

other inflammatory conditions associated with particular organs or tissues, such as nephritis (e.g., glomerulonephritis)—oliguria, abnormal urinalysis;

inflamed appendix—fever, pain, tenderness, leukocytosis;

gout—pain, tenderness, swelling and erythema of the involved joint, elevated serum and/or urinary uric acid;

inflamed gall bladder—abdominal pain and tenderness, fever, nausea, leukocytosis;

chronic obstructive pulmonary disease—shortness of breath, wheezing;

congestive heart failure—shortness of breath, rales, peripheral edema;

Type II diabetes—end organ complications including cardiovascular, ocular, renal, and peripheral vascular disease, lung fibrosis—hyperventilation, shortness of breath, decreased oxygenation;

vascular disease, such as atherosclerosis and restenosis—pain, loss of sensation, diminished pulses, loss of function and alloimmunity leading to transplant rejection—pain, tenderness, fever.

Subclinical symptoms include without limitation diagnostic markers for inflammation the appearance of which may precede the manifestation of clinical symptoms. One class of subclinical symptoms is immunological symptoms, such as the invasion or accumulation in an organ or tissue of proinflammatory lymphoid cells or the presence locally or peripherally of activated pro-inflammatory lymphoid cells recognizing a pathogen or an antigen specific to the organ or tissue. Activation of lymphoid cells can be measured by techniques known in the art.

### Definitions

Unless defined otherwise, all technical and scientific terms used herein generally have the same meaning as commonly understood by one of ordinary skill in the art to which this invention belongs.

The term “amorphous” as used herein, when applied to an active ingredient, means that the active ingredient is not completely crystalline, e.g., present in a poorly crystalline, partially crystalline, semi-crystalline, non-crystalline, partially amorphous or partially disordered form.

The term “about” or “approximately” as used herein means within an acceptable error range for the particular value as determined by one of ordinary skill in the art, which will depend in part on how the value is measured or determined, i.e., the limitations of the measurement system. For example, “about” can mean within 1 or more than 1 standard deviations, per the practice in the art. Alternatively, “about” can mean a range of up to 20%, and preferably up to 10% of a given value. Alternatively, particularly with respect to biological systems or processes, the term can mean within an order of magnitude, preferably within 5-fold, and more preferably within 2-fold, of a value.

The term “bioavailability” refers to the rate and extent to which the active ingredient or active moiety is absorbed from a drug product and becomes systematically available.

The term “effective amount” means the amount of the formulation, which when administered to a patient (e.g., a mammal) for treating a disease, contains sufficient active ingredient to effect such treatment for the disease, or an amount that is sufficient for inhibiting phosphodiesterase (such as PDE4) or increasing cyclic AMP levels, so as to achieve the objectives of the invention. The “effective amount” will vary depending on the compound, the disease and its severity and the age, weight, physical condition and responsiveness, etc., of the patient to be treated.

The term “retard” formation as used herein means to slow, inhibit, reduce, hinder, impede or delay formation.

The term “substantially pure” as used herein, when applied to the amorphous form of the active ingredient, means that greater than about 10% of the active ingredient is amorphous, for example greater than about 20%, about 40%, greater than about 60%, greater than about 65%, greater than about 70%, greater than about 75%, greater than about 80%, greater than about 85%, greater than about 90%, greater than about 95%, greater than about 97.5%, greater than about 98%, greater than about 99%, or greater than about 99.5% of the active ingredient is amorphous.

The pharmacokinetic parameters described herein include area under the plasma vs. concentration-time curve during the dosing interval, τ (AUC_{0-τ}), area under the plasma vs. concentration-time curve from time zero up to the time corresponding to the last measurable plasma concentration (AUC₀₋ₜ), maximum plasma concentration (Cₘₐₓ), average steady-state plasma concentration (Cₐᵥ), time of maximum plasma concentration (Tₘₐₓ) and terminal elimination half-life (T_{1/2}). The time of maximum concentration, T_{max,} is determined as the time corresponding to Cₘₐₓ.

Area under the plasma concentration-time curve up to the time corresponding to the last measurable concentration (AUC₀₋ₜ) is calculated by numerical integration using the linear trapezoidal rule as follows:

$${AUC}_{0 - t} = {\sum }_{i = 2}^{n} 0.5 * ( {C}_{i} + {C}_{i - 1} ) * ( {t}_{i} - {t}_{i - 1} ) \qquad Eq . 1$$

where Cᵢ is the plasma oglemilast concentrations at the corresponding sampling time point tᵢ and n is the number of time points up to and including the last quantifiable concentration. AUC_{0-τ} is calculated using Equation 1 with t=τ (24 hours)

The terminal half-life (T_{1/2}) is calculated using the following equation:

$${T}_{1 / 2} = \frac{0.693}{{λ}_{z}} \qquad Eq . 2$$

where λ_{z} is the terminal elimination rate constant determined by performing a regression analysis on the terminal linear phase of semilogarithmic plots of individual oglemilast concentration-time data using noncompartmental analysis in WinNolin version 4.1.

The area under the plasma concentration-time curve from time zero to infinity is calculated according to the following equation:

$${AUC}_{0 - ∞} = {AUC}_{0 - t} + \frac{{C}_{last}}{{λ}_{z}} \qquad Eq . 3$$

where Cₗₐₛₜ is the last measurable concentration.

Cₐᵥ is determined using the following equation:

Cₐᵥ=AUC_{0-τ}/τ Eq. 4

The terms “treat,” “treatment,” and “treating” refer to one or more of the following:
(a) relieving or alleviating at least one symptom of a disorder in a subject, including for example, allergic and inflammatory disorders, such as asthma and COPD;
(b) relieving or alleviating the intensity and/or duration of a manifestation of a disorder experienced by a subject including, but not limited to, those that are in response to a given stimulus (e.g., pressure, tissue injury, cold temperature, etc.);
(c) arresting, delaying the onset (i.e., the period prior to clinical manifestation of a disorder) and/or reducing the risk of developing or worsening a disorder.

A subject or patient in whom administration of the therapeutic compound is an effective therapeutic regimen for a disease or disorder is preferably a human, but can be any animal, including a laboratory animal in the context of a clinical trial or screening or activity experiment. Thus, as can be readily appreciated by one of ordinary skill in the art, the methods, compounds and compositions of the present invention are particularly suited to administration to any animal, particularly a mammal, and including, but by no means limited to, humans, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, etc., avian species, such as chickens, turkeys, songbirds, etc., i.e., for veterinary medical use.

### EXAMPLES

The present invention will now be further described by way of the following non-limiting examples. In applying the disclosure of these examples, it should be kept clearly in mind that the examples are merely illustrative of the present invention and should not be construed as limiting the scope of the invention in any way as many variations and equivalents that are encompassed by the present invention will become apparent to those skilled in the art upon reading the present disclosure.

In the foregoing and in the following examples, all temperatures are set forth uncorrected in degrees Celsius; and, unless otherwise indicated, all parts and percentages are by weight.

The entire disclosures of all applications, patents and publications, cited above and below, are hereby incorporated by reference.

### Example 1

The present example compares the results of administration to 17 humans at a dose of 12 mg active ingredient per subject of (i) two tablet formulations of the present invention, both prepared by wet granulation (Formulations I and II) and (ii) conventional tablets prepared by direct compression (Formulation III) and a conventional dry powder suspension (Formulation IV).

### Formulations I and II

TABLE 1
Ingredients for Formulations I and II

| | | | % |
|---|---|---|---|
| Ingredients | Functionality | mg/tablet | (w/w) |
| Oglemilast sodium | Active | 12.0 | 5.45 |
| Povidone, USP | Binder | 12.0 | 5.45 |
| Silicified Microcrystalline Cellulose* | Diluent | 90.0 | 40.90 |
| Sodium Starch Glycolate, NF | Disintegrant | 14.4 | 6.53 |
| Silicified Microcrystalline Cellulose | Diluent | 81.5 | 37.06 |
| Colloidal Silicon Dioxide, NF | Glidant | 4.2 | 1.91 |
| Talc, USP | Glidant | 4.2 | 1.91 |
| Magnesium Stearate, NF | Lubricant | 1.7 | 0.79 |
| Purified Water USP** | Solvent | 0.0 | 0 |
| Tablet, 12 mg (Formulations I and II) | — | 220.0 | 100 |

**Water is removed during formulation

### Process I—Preparation of Formulation I

Stage 1a—The silicified microcrystalline cellulose (*) and about half of the sodium starch glycolate were premixed and preheated in a fluid bed. Oglemilast sodium salt and povidone were dispersed in water.

Stage 2—The preheated mixture from stage 1a was granulated using the dispersion at a temperature less than 60° C. The co-processed granules were then dried to a constant loss on drying (LOD) amount of about less than 6% at about less than 80° C. in the fluid bed.

### Process II—Preparation of Formulation II

Stage 1b—The silicified microcrystalline cellulose (*) and about half of the sodium starch glycolate and oglemilast sodium salt were premixed and preheated in a fluid bed. A solution (solution 1b) of povidone was prepared in water.

Stage 2—The preheated mixture from stage 1b was granulated using solution 1b at a temperature less than 60° C. The co-processed granules were then dried to a constant loss on drying (LOD) amount of about less than 6% at about less than 80° C. in the fluid bed

In each Process, the dried granules from stage 2 were blended with the remaining portions of sodium starch glycolate silicified microcrystalline cellulose, colloidal silicon dioxide and talc in a V-blender. The final blend was prepared by mixing the blended material with magnesium stearate in a V-blender and then compressed into tablets.

### Formulation III

TABLE 2
Ingredients for Formulation III

| Ingredient | Functionality | mg/Tab | % (w/w) |
|---|---|---|---|
| Oglemilast sodium | Active | 12.0 | 3.20 |
| Microcrystalline Cellulose, NF | Diluent | 60.0 | 16.00 |
| Dibasic Calcium Phosphate, | Diluent | 187.0 | 49.87 |
| Dihydrate, NF | | | |
| Pregelatinized Starch, NF | Binder | 20.0 | 5.33 |
| Sodium Bicarbonate, USP | Alkalizer/diluent | 40.0 | 10.67 |
| Magnesium Oxide, USP Heavy, | Alkalizer/diluent | 10.0 | 2.67 |
| Powder, | | | |
| Povidone, USP | Binder | 5.0 | 1.33 |
| Crospovidone, NF | Disintegrant | 24.0 | 6.40 |
| Croscarmellose Sodium, NF | Disintegrant | 11.0 | 2.93 |
| Colloidal Silicon Dioxide, NF | Glidant | 2.0 | 0.53 |
| Magnesium Stearate, NF | Lubricant | 4.0 | 1.07 |
| Tablets, 12 mg (Formulation III) | — | 375.0 | 100 |

Oglemilast sodium salt and microcrystalline cellulose were blended in a V-blender with prescreened dibasic calcium phosphate dihydrate, pregelatinized starch, sodium bicarbonate, magnesium oxide, povidone, crospovidone, croscarmellose sodium and colloidal silicon dioxide. The mixture was blended with prescreened magnesium stearate to generate the final blend, which was then compressed into tablets.

### Formulation IV

TABLE 3
Ingredients for Formulation IV

| | Amount |
|---|---|
| Ingredients | (w/w) |
| Oglemalast sodium | 0.315 |
| Sodium lauryl sulfate, USP | 1.50 |
| Povidone, USP | 1.50 |
| Mannitol, USP | 19.46 |
| Mannitol, USP | 74.25 |
| Xanthan gum, FNCS, Food Grade* | 0.7 |
| Sodium Saccharin, USP | 0.15 |
| Sodium Benzoate, NF | 1.2 |
| Artificial Strawberry Flavor | 1.00 |
| Colloidal Silicon Dioxide, NF | 0.15 |
| Total (Formulation IV) | 100 |

The oglemilast sodium salt and sodium lauryl sulphate were triturated. Povidone was added to the active triturate and mixed. This mixture was then triturated with xanthan gum, saccharin sodium, sodium benzoate and strawberry flavor. The triturated mixture was then transferred to a blender and mannitol was then blended into the mixture. The blend was discharged and sieved with colloidal silicon dioxide. All the ingredients were then blended together.

The dissolution rates of the active ingredient in Formulations I-IV are shown in Table 4. Dissolution rates for the active ingredient in Formulations I-III were determined using USP Apparatus II (paddles) at 50 RPM with 0.1 N HCl, 1%-2% sodium dodecyl sulfate at 5, 15, 30, 45, and 60 minute sampling intervals (Formulation I-III). The dissolution rate of the active ingredient in the conventional dry powder suspension (Formulation IV) was determined using 12 mg of API equivalent of the dry powder suspension (in an aqueous slurry) using USP Apparatus II (paddles) at 50 RPM with 0.1 N HCl, 1%-2% sodium dodecyl sulfate at 5, 15, 30, 45, and 60 minute sampling intervals.

TABLE 4
Dissolution Rates

| | | Formulation | Formulation | Formulation |
|---|---|---|---|---|
| | Formulation I | II | III | IV |
| Time (min) | % Dissolved | % Dissolved | % Dissolved | % Dissolved |
| 5 | 79 | 67 | 29 | 77 |
| 15 | 86 | 77 | 42 | 79 |
| 30 | 90 | 82 | 51 | 81 |
| 45 | 91 | 85 | 59 | 81 |
| 60 | 92 | 86 | 64 | 82 |

As can be seen from Table 4, tablet Formulations I and II of the present invention show significantly superior dissolution rates when compared to tablet conventional Formulation III.

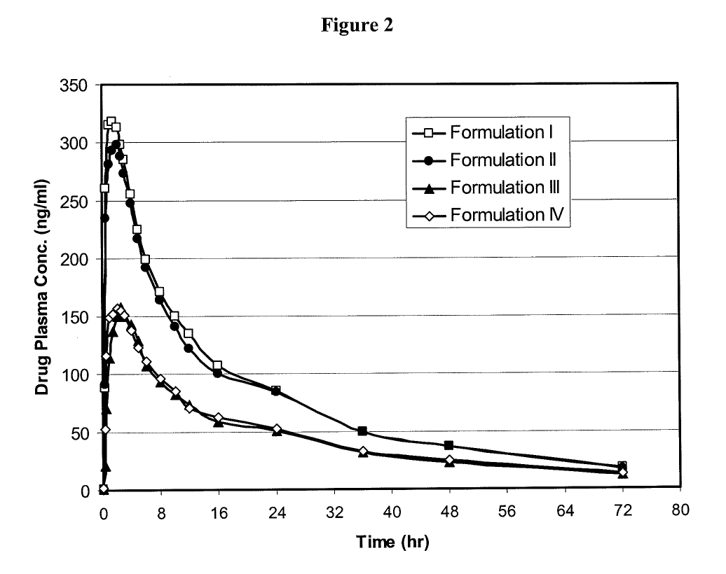

The mean in vivo pharmacokinetic parameters following oral administration of Formulations I-IV to 17 humans at a dose of 12 mg active ingredient per subject are shown in Table 5 and in FIG. 2.

TABLE 5
Mean pharmacokinetic parameters for Formulations I-IV

| | Tₘₐₓ | Cₘₐₓ | AUC_{0-inf} | T_{1/2} |
|---|---|---|---|---|
| | (hr) | (ng/ml) | (ng hr/ml) | (hr) |
| Formu- | 1.9 ± 1.3 | 353.2 ± 123 | 6000 ± | 21.8 ± 7.8 |
| lation I | 1.5 (0.5-5.0)* | | 1784 | 19.6 (13.4-38.3)* |
| Formu- | 2.4 ± 2.1 | 321.8 ± 96 | 5744 ± | 20.6 ± 5.4 |
| lation II | 2.0 (0.5-10.0)* | | 1637 | 20.6 (14.3-34.5)* |
| Formu- | 2.5 ± 1.2 | 187.9 ± 34 | 3736 ± | 21.6 ± 7.2 |
| lation | 2.0 (0.5-5.0)* | | 1206 | 20.8 (11.9-43.7)* |
| III | | | | |
| Formu- | 2.4 ± 1.2 | 165.9 ± 55 | 3564 ± | 23.4 ± 9.5 |
| lation | 2.5 (1.0-5.0)* | | 1495 | 21.5 (13.3-52.2)* |
| IV | | | | |

*median range

As can be seen from Table 5 and FIG. 2, tablet formulations of the present invention (Formulations I and II) yielded comparable systemic exposures that were significantly higher than observed for the conventional tablet (Formulation III) and the conventional dry powder suspension (Formulation IV).

### Example 2

This Example shows that conventional formulations containing crystalline oglemilast sodium exhibit low-bioavailability.

A conventional dry powder containing crystalline oglemilast sodium salt was prepared by mixing the ingredients set forth below in Table 6:

TABLE 6
Ingredients for conventional dry powder

| Ingredient | % (w/w) |
|---|---|
| Oglemilast sodium | 0.315 |
| Sodium lauryl sulphate | 1.50 |
| Polyvinyl pyrrolidone (Kollidon 30) | 1.50 |
| Mannitol (D-mannitol 25) | 19.46 |
| Mannitol (Pearlitol SD-200) | 74.00 |
| Xanthan gum | 0.700 |
| Carmosine color | 0.025 |
| Sodium saccharin | 0.150 |
| Sodium benzoate | 1.200 |
| Strawberry flavor | 1.000 |
| Colloidal anhydrous silica (Aerosil 200) | 0.150 |
| Dry Powder | 100 |

The oglemilast sodium salt had a particle size distribution characterized by X₉₀ greater than about 10 μm.

A liquid suspension of the above dry powder (3 mg/g) was prepared in water and administered to humans as a single dose of 1, 3, 6, 12, or 18 mg active ingredient, or at a dose of 3, 9, 15, or 24 mg active ingredient per day for multiple days. Mean pharmacokinetic data is provided in Tables 7 and 8:

TABLE 7
Single Dose Administration

| | Daily Dose | | | | |
|---|---|---|---|---|---|
| | 1 mg | 3 mg | 6 mg | 12 mg | 18 mg |
| AUC₀₁₋₁₂ | 156 | 442 | 550 | 793 | 971 |
| (ng hr/mL) | | | | | |
| Cₘₐₓ (ng/mL) | 19 | 51 | 77 | 107 | 135 |
| Tₘₐₓ (hr) | 3 | 2.3 | 1 | 1.5 | 2 |

TABLE 8
Multiple Dose Administration
(pharmacokinetic parameters are for day 1 of the administration)

Daily Dose

| | 3 mg | 9 mg | 15 mg | 24 mg |
|---|---|---|---|---|
| AUC₀₁₋₁₂ | 444 | 814 | 882 | 1385 |
| (ng hr/mL) | | | | |
| Cₘₐₓ (ng/mL) | 44 | 87 | 121 | 129 |
| Tₘₐₓ (hr) | 1.5 | 1.8 | 1.3 | 1.8 |

As can be seen from Tables 7 and 8, a conventional dry powder suspension formulation containing crystalline oglemilast sodium exhibits low bioavailability.

### Example 3

The present Example describes the results of oral administration of (i) a conventional solution of oglemilast sodium salt in water, ethanol and polyethylene glycol 400 (Formulation A), (ii) conventional capsules containing oglemilast sodium salt (Formulations B and C) and (iii) conventional capsules containing crystalline oglemilast (Formulation D) to 3 male beagle dogs.

### Formulation A—Preparation of a Conventional Solution from Oglemilast Sodium

10 mL ethyl alcohol was added to 100 mg oglemilast sodium salt and the resulting mixture was stirred until a clear solution was obtained. 22.5 mL polyethylene glycol 400 was added and mixed for 5 minutes. 67.5 mL purified water was added and mixed for 5 minutes. The resulting solution (1 mg/mL) was filtered through a 0.22 micron filter and dosed at 1 mg/kg.

### Formulations B and C—Preparation of Conventional Capsules Containing Crystalline Oglemilast Sodium

A size 0 capsule shell was weighed and then filled with oglemilast sodium salt powder to obtain an equivalent to 2 mg/kg of animal weight of active ingredient in the capsule. The capsule was closed and a gross weight determined. The net weight of the oglemilast sodium salt being delivered was then calculated. Formulations B and C contained crystalline oglemilast sodium obtained from two different production runs.

### Formulation D—Preparation of Conventional Capsules Containing Crystalline Oglemilast

Preparation of crystalline oglemilast: Oglemilast sodium salt was dispersed in 0.1N HCl and mixed by sonication for one hour. The resulting solid was collected by filtration. The solid was then refluxed in methanol at a temperature less than about 80° C. for several hours. The resulting solid was collected by filtration and dried. XRD and FTIR confirmed the identity of crystalline oglemilast. The absence of sodium was confirmed by elemental analysis.

Capsule preparation: A size 0 capsule shell was weighed and then filled with oglemilast to obtain an equivalent to 2 mg/kg of animal weight of active ingredient in the capsule. The capsule was closed and a gross weight determined. The net weight of the oglemilast being delivered was then calculated.

Mean Cₘₐₓ profiles for each Formulation A-D are provided in Table 9 below. Mean plasma pharmacokinetic parameters are set forth in Table 10.

TABLE 9
Mean plasma concentration-time data

| | | Time (hr) | | | | | | | | | |
|---|---|---|---|---|---|---|---|---|---|---|---|
| | | 0.00 | 0.50 | 1.00 | 1.50 | 2.00 | 3.00 | 4.00 | 8.00 | 12.00 | 24.00 |
| Formulation A | Mean | 0.00 | 72.04 | 108.68 | 108.56 | 99.90 | 87.11 | 78.31 | 50.48 | 25.21 | 7.81 |
| Oral Solution | SD | 0.00 | 5.35 | 24.97 | 13.61 | 6.58 | 7.92 | 10.19 | 17.64 | 24.51 | 13.53 |
| (1 mg/mL) | | | | | | | | | | | |
| Dosed at 1 mg/kg | | | | | | | | | | | |
| Formulation B | Mean | 0.00 | 130.11 | 519.55 | 450.85 | 424.36 | 333.86 | 279.16 | 163.90 | 93.52 | 28.52 |
| Oral Capsule | SD | 0.00 | 88.61 | 436.33 | 322.23 | 277.59 | 199.35 | 176.73 | 105.99 | 60.00 | 26.98 |
| Dosed at 2 mg/kg | | | | | | | | | | | |
| Formulation C | Mean | 0.00 | 264.20 | 554.48 | 488.23 | 461.48 | 322.97 | 250.59 | 119.09 | 76.04 | 24.76 |
| Oral Capsule | SD | 0.00 | 223.20 | 692.21 | 603.31 | 445.02 | 335.85 | 271.83 | 157.68 | 105.13 | 42.89 |
| Dosed at 2 mg/kg | | | | | | | | | | | |
| Formulation D | Mean | 0.00 | 0.00 | 0.00 | 0.00 | 0.00 | 0.00 | 7 | 8 | 0.00 | 0.00 |
| Oral Capsule | SD | 0.00 | 0.00 | 0.00 | 0.00 | 0.00 | 0.00 | ND | ND | 0.00 | 0.00 |
| Dosed at 2 mg/Kg | | | | | | | | | | | |

TABLE 10
Mean plasma pharmacokinetic parameters

| | | Cₘₐₓ | | AUC_{0-24 hr} |
|---|---|---|---|---|
| | | (ng/mL) | Tₘₐₓ (hr) | (ng · hr/mL) |
| Formulation A | Mean | 233.48 | 1.33 | 1758.74 |
| Oral Solution | SD | 25.34 | 0.58 | 767.62 |
| 1 mg/mL | | | | |
| Calculated at 2 mg/Kg | | | | |
| Formulation B | Mean | 545.78 | 1.33 | 3492.08 |
| Oral Capsule | SD | 399.92 | 0.58 | 2256.84 |
| 2 mg/kg | | | | |
| Formulation C | Mean | 699.93 | 1.17 | 3075.91 |
| Oral Capsule | SD | 570.58 | 0.76 | 3784.02 |
| 2 mg/kg | | | | |
| Formulation D | Mean | 6.9 | Not | Not |
| Oral Capsule | | | Calculated. | Calculated. |
| 2 mg/kg | | | | |

As can be seen, a conventional solution formulation prepared from crystalline oglemilast sodium (Formulation A) and conventional capsule formulations containing crystalline oglemilast sodium (Formulations B and C) exhibit low-bioavailability. A conventional capsule formulation containing crystalline oglemilast exhibits essentially no bioavailability.

### Example 4

The present Example describes the results of oral administration to male beagle dogs of (i) 12 mg tablets of Formulation I of the present invention (see Example 1), (ii) a solution formulation of the present invention prepared from oglemilast sodium salt (100 mg), PEG 400 (20 g), ethanol (25 g), povidone (Kollidon 30) (200 mg), sodium hydroxide (0.1 N solution 1.5 ml) and water (q.s. 100 ml), (iii) a conventional solution of oglemilast sodium salt in water, ethanol and PEG 400 (1 mg/kg), and (iv) a conventional dry powder suspension (Formulation IV, see Example 1) (normalized to a 12 mg dose).

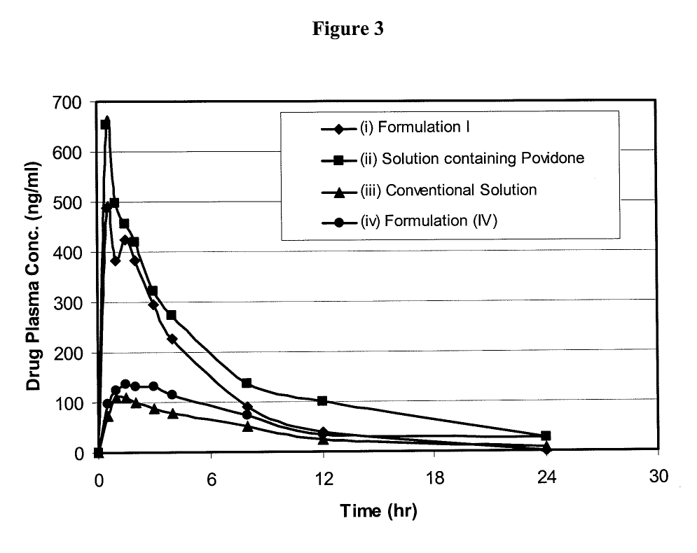

As can be seen from FIG. 3, the conventional solution (iii) and the conventional dry powder suspension (iv) have much lower mean plasma concentrations than either the tablet formulation of the present invention (i) or the solution formulation of the present invention (ii).

### Example 5

The present Example describes effect of the particle size of the active ingredient, and describes the results of oral administration of two conventional dry powder suspensions of oglemilast sodium to humans at a dose of 12 mg, one with the active drug particle size having X₉₀ greater than 10 microns (Formulation E) and the other with the active drug particle size having X₉₀ less than 10 microns (Formulation F).

The two conventional dry powder suspensions were prepared by mixing the ingredients shown in Table 11. Each powder was mixed with water to prepare a liquid suspension for administration.

TABLE 11
Ingredients for Formulations E and F

| | Formulation E | Formulation F |
|---|---|---|
| Component | % (w/w) | % (w/w)) |
| oglemilast sodium | 0.315 | 0.315 |
| Sodium lauryl sulphate | 1.50 | 1.50 |
| Polyvinyl pyrrolidone | 1.50 | 1.50 |
| Mannitol (D-Mannitol 25) | 19.46 | 19.46 |
| Mannitol (SD-200) | 74.000 | 74.025 |
| Xanthan gum | 0.700 | 0.700 |
| Carmosine color | 0.025 | 0.00 |
| Sodium saccharin | 0.150 | 0.150 |
| Sodium benzoate | 1.200 | 1.200 |
| Strawberry flavor | 1.000 | 1.000 |
| Colloidal anhydrous silica | 0.150 | 0.150 |
| Dry Powder Suspension, | 100.0 | 100.0 |
| (3 mg/g) | | |

Representative particle size characteristics of the active ingredient used in Formulations E and F are presented in Table 12.

TABLE 12
Particle size characteristics of the active ingredient

| | |
|---|---|
| Formulation E | Formulation F |

| | | | |
|---|---|---|---|
| X₁₀ | 0.8 μm | X₁₀ | 0.7 μm |
| X₅₀ | 4.1 μm | X₅₀ | 1.9 μm |
| X₉₀ | 36.9 μm | X₉₀ | 7.0 μm |

Dissolution properties of the active ingredient in Formulations E and F are presented in Table 13.

TABLE 13
Dissolution properties

| | |
|---|---|
| Formulation E | Formulation F |

| Time (min) | % dissolved | Time (min) | % dissolved |
|---|---|---|---|
| 0 | 0 | 0 | 0 |
| 5 | 46.7 | 5 | 99.0 |
| 15 | 55.9 | 15 | 97.9 |
| 30 | 62.5 | 30 | 98.1 |
| 60 | 68.8 | 60 | 98.2 |

Mean pharmacokinetic parameters for Formulations E and F, administered to humans at a dose of 12 mg, are presented in Table 14.

TABLE 14
Mean pharmacokinetic parameters

| | Formulation E | Formulation F |
|---|---|---|
| AUC (ng hr/mL) | 793 | 3546 |
| Cₘₐₓ (ng/mL) | 107 | 166 |
| Tₘₐₓ (hours) | 1.5 | 2.4 |

As can be seen, the use of oglemilast sodium with particle size having X₉₀ less than 10 microns results in a formulation (Formulation F) that has approximately twice the dissolution rate of Formulation E (having an oglemilast sodium particle size with X₉₀ greater than 10 microns) and also provides approximately a 4-fold increase in bioavailability (AUC), when compared to Formulation E.

### Example 6

The present Example described the results of oral administration to beagle dogs of two different solution formulations prepared from crystalline oglemilast sodium: (i) a conventional solution formulation (Formulation G), and (ii) a solution formulation of the present invention that also contains polyvinyl pyrrolidone (Formulation H).

### Formulation G

A solution was prepared by mixing 100 mg oglemilast sodium, 10 mL ethyl alcohol, 22.5 mL polyethylene glycol 400 and 67.5 mL purified water.

### Formulation H

A solution was prepared by mixing 100 mg oglemilast sodium, 25 g ethyl alcohol, 25 g polyethylene glycol 400, 200 mg polyvinyl pyrrolidone, 1.5 mL sodium hydroxide (0.1 N solution) and purified water (q.s. 100 mL).

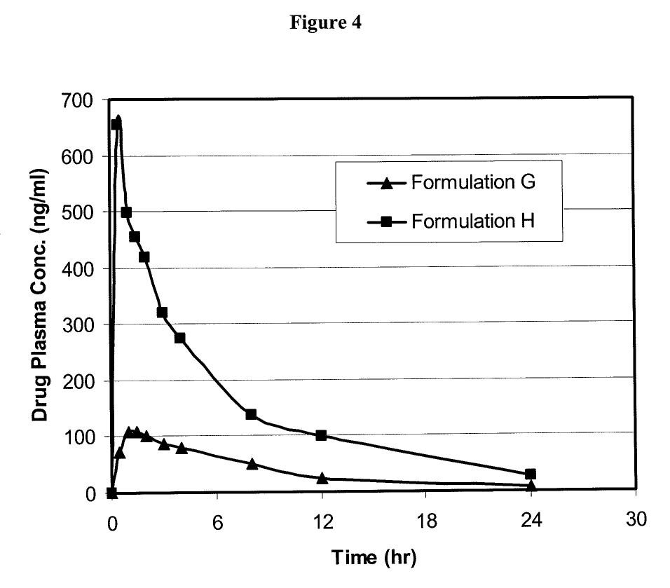

The mean plasma profiles for Formulations G and H, orally administered as 1 mg/ml concentrated solutions to 3 male beagle dogs at a dose of 12 mg are shown in FIG. 4. As can be seen, solution Formulation H of the present invention has a much higher mean plasma concentration than conventional solution Formulation G.

### Example 7

The present Examples describes the results of oral administration to dogs of (i) a 12 mg tablet of the present invention containing amorphous form of the active drug prepared using oglemilast sodium salt (Formulation J), and (ii) a 12 mg tablet of the present invention containing amorphous form of the active drug prepared using oglemilast (Formulation K).

### Preparation of Formulation J

Oglemilast sodium salt and polyvinyl pyrrolidone (1:2 ratio) were dissolved in a mixture of ethanol and ammonium hydroxide. The solution was then dried in vacuo and the solid thus obtained was formulated into tablets according to Table 15:

TABLE 15
Ingredients for Formulation J

| Ingredient | % w/w |
|---|---|
| Oglemilast sodium salt/PVP mixture | 18.2 |
| Sodium Starch Glycolate, NF | 6.54 |
| Silicified Microcrystalline Cellulose | 70.67 |
| Colloidal Silicon Dioxide, NF | 1.91 |
| Talc | 1.91 |
| Magnesium Stearate | 0.77 |
| Total | 100 |

### Preparation of Formulation K

Oglemilast (23.25% w/w), polyvinyl pyrrolidone (69.77% w/w) and sodium chloride (6.98% w/w) were dissolved in a mixture of ethanol and ammonium hydroxide. The solution was then dried in vacuo and the solid thus obtained was formulated into tablets according to Table 16:

TABLE 16
Ingredients for Formulation K

| Ingredient | % w/w |
|---|---|
| Oglemilast/PVP mixture | 20.23 |
| Sodium Starch Glycolate, NF | 6.54 |
| Silicified Microcrystalline Cellulose | 68.64 |
| Colloidal Silicon Dioxide, NF | 1.91 |
| Talc | 1.91 |
| Magnesium Stearate | 0.77 |
| Total | 100 |

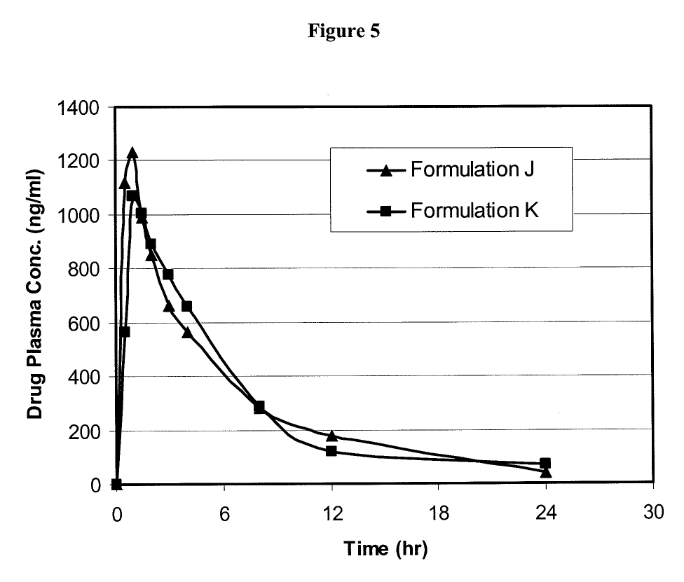

FIG. 5 shows the plasma concentrations of Formulations J and K following oral administration to dogs at a dose of 12 mg. As can be seen, Formulations J and K (both containing amorphous forms of the active ingredient prepared using oglemilast sodium salt and oglemilast, respectively) exhibit high bioavailabilities.

### Example 7

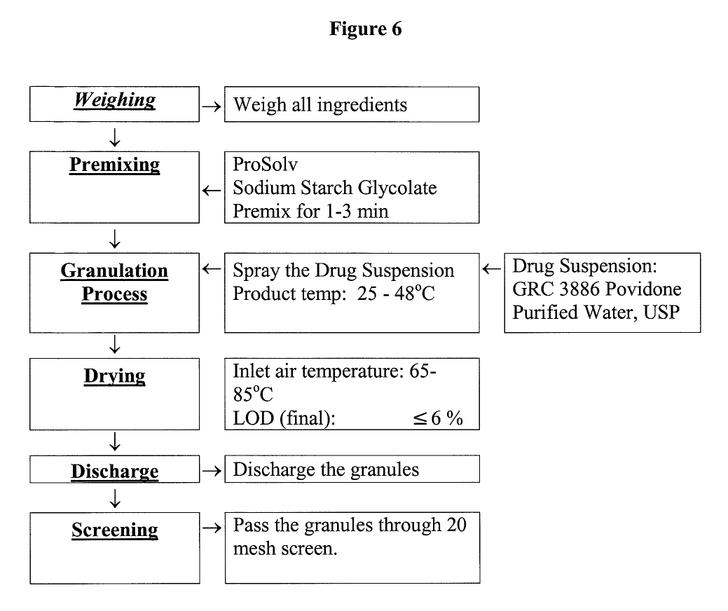

Oglemilast granules were prepared in accordance with the granulation process shown in FIG. 6 (fluid bed: GPCG 3.1, top spray, atomization pressure: 1.5 to 1.8 bar, inlet air flow: 15 to 150 CFM, shake mechanism: asynchronous). The composition of the granules is shown in Table 17.

TABLE 17
Composition of Oglemilast Granules

| | Granule Strength (mg/g) | | | | | | | |
|---|---|---|---|---|---|---|---|---|
| | 20 mg/g | | 40 mg/g | | 80 mg/g | | 100 mg/g | |
| Ingredients | Amount (grams per batch) | | | | | | | |
| Oglemilast Sodium | 36 | | 80 | | 160 | | 150 | |
| Povidone | 36 | | 80 | | 160 | | 150 | |
| Microcrystalline | 1692 | | 1760 | | 1520 | | 1110 | |
| Cellulose/Colloidal | | | | | | | | |
| Silicon Dioxide | | | | | | | | |
| (ProSolv) | | | | | | | | |
| Sodium Starch Glycolate | 36 | | 80 | | 160 | | 90 | |
| Purified water* | 1046 | | 2000 | | 3000 | | 4350 | |
| Total weight of granules | 1800 | g | 2000 | g | 2000 | g | 1500 | g |
| (g) | | | | | | | | |

*Purified water is evaporated during processing.

### Example 8

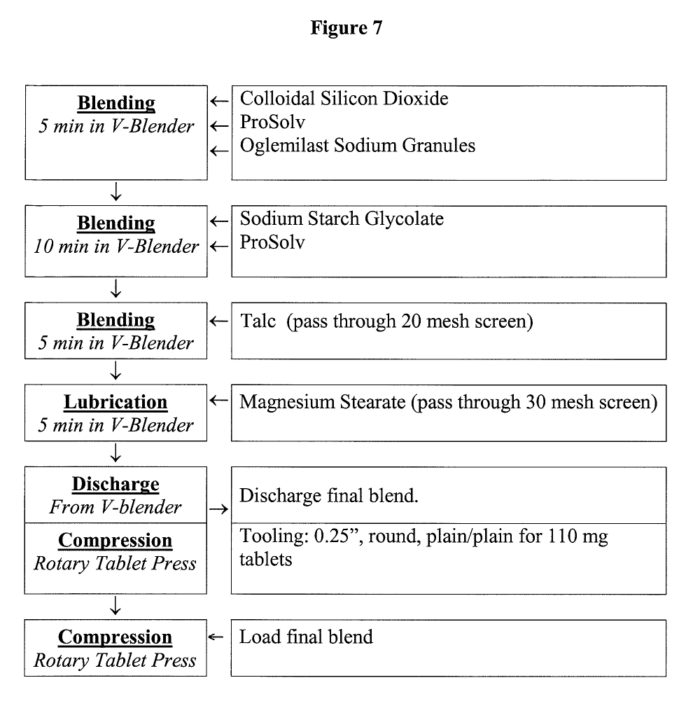

Oglemilast tablets were prepared in accordance with the process shown FIG. 7. The composition of the tablets is shown in Table 18. The compression parameters for the 110 mg tablets were as follows: average wt. of 10 tablets: 1.07 to 1.14 g, individual hardness: 2 to 6 kp.

TABLE 18
Compositions of Oglemilast Tablets

| Strength | | | |
|---|---|---|---|
| 0.1 mg | 0.6 mg | 1.25 mg | 2.5 mg |

| | |
|---|---|
| Ingredient | Weight (grams per batch) |

Blend

| Oglemilast Sodium | 50 | | 300 | | — | | — | |
|---|---|---|---|---|---|---|---|---|
| Granules, 20 mg/g | | | | | | | | |
| Oglemilast Sodium | — | | — | | 125 | | 250 | |
| Granules, 100 mg/g | | | | | | | | |
| Colloidal Silicon Dioxide | 11 | | 11 | | 11 | | 11 | |
| Sodium Starch Glycolate | 50 | | 50 | | 50 | | 50 | |
| Microcrystalline | 954 | | 704 | | 879 | | 754 | |
| Cellulose/Colloidal | | | | | | | | |
| Silicon Dioxide | | | | | | | | |
| (ProSolv) | | | | | | | | |
| Talc | 30 | | 30 | | 30 | | 30 | |
| Magnesium Stearate, | 5 | | 5 | | 5 | | 5 | |
| Total weight of Final | 1100 | g | 1100 | g | 1100 | g | 1100 | g |
| Blend (grams) | | | | | | | | |

Compression

| | | | | | | | | |
|---|---|---|---|---|---|---|---|---|
| Tablets weight (mg) | 110 | mg | 110 | mg | 110 | mg | 110 | mg |

### Example 9

### A Single-Center, Double-Blind, Randomized, Placebo-Controlled Parallel-Group 7-Day Multiple Dose Study to Evaluate the Safety, Tolerability and Pharmacokinetics of Oglemilast in Healthy Subjects

The primary objective of this study was to demonstrate the safety, tolerability and pharmacokinetics of multiple doses (0.1, 0.6, 1.25 and 2.5 mg) of tablet formulations of oglemilast. The composition of the tablets is given in Table 18.

### Methodology

This was a single-center, randomized, double-blind, placebo-controlled parallel-group 7-day multiple dose study which enrolled 30 healthy male and female subjects, 18 to 45 years of age. The subjects were randomized to receive one of the five following treatments (6 subjects per treatment group):

Treatment A: Multiple oral doses of 0.1 mg oglemilast (1×0.1 mg tablet) once a day for 7 days

Treatment B: Multiple oral doses of 0.6 mg oglemilast (1×0.6 mg tablet) once a day for 7 days

Treatment C: Multiple oral doses of 1.25 mg oglemilast (1×1.25 mg tablet) once a day for 7 days

Treatment D: Multiple oral doses of 2.5 mg oglemilast (1×2.5 mg tablet) once a day for 7 days

Treatment E: Multiple oral doses of matching placebo (1 tablet) once a day for 7 days

The subjects received the study drug at 0800 hours with 240 mL of water on Days 1 to 7. The subjects underwent a 10 hour fasting period before each dosing. Following each dose, the subjects continued their fast and remained seated upright and awake for 4 hours.

Blood samples were collected for PK analysis as follows:

Day 1: 0.0 (predose), 0.25, 0.5, 1, 1.5, 2, 2.5, 3, 4, 5, 6, 8, 10, 12, 16 and 24 hours postdose;

Days 5 and 6: 0.0 (predose) hours;

Day 7: 0.0 (predose), 0.25, 0.5, 1, 1.5, 2, 2.5, 3, 4, 5, 6, 8, 10, 12, 16, 24, 26, 48 and 72 hours postdose.

The mean in vivo pharmacokinetic parameters following oral administration of the tablet formulations are presented in Table 19.

TABLE 19
Mean PK Steady State Parameters

| | Cₘₐₓ | AUC₀₋₂₄ | Tₘₐₓ | T_{1/2} |
|---|---|---|---|---|
| Strength | ng/mL | ng · hr/mL | hr | hr |
| 0.1 mg (100 mcg) | 8.7 | 124.8 | 2.5 | 19.1 |
| 0.6 mg (600 mcg) | 48.3 | 626.3 | 1.6 | 19.4 |
| 1.25 mg (1250 mcg) | 114.6 | 1418 | 1.7 | 18.9 |
| 2.5 mg (2500 mcg) | 217.8 | 2831 | 1.2 | 20.1 |

The mean calculated Tₘₐₓ value is 1.75 hours and the mean calculated T_{1/2} value is 19.4 hours.

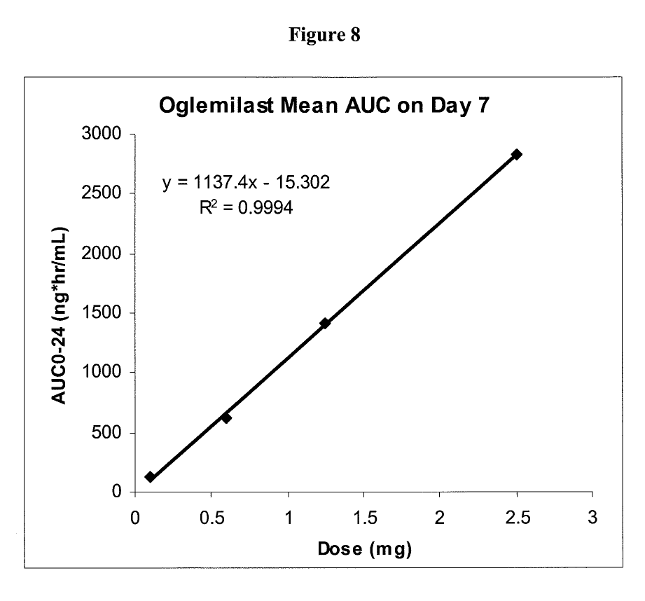

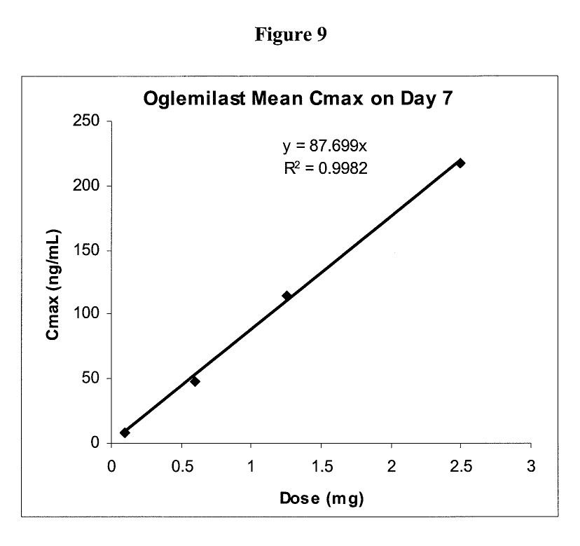

FIG. 8 shows the linear regression for mean AUC₀₋₂₄ and dose values given in Table 19. FIG. 9 shows the linear regression for mean Cₘₐₓ and dose values given in Table 19.

Table 20 shows the calculated mean steady state PK parameters, based on a linear regression results shown in Table 19.

TABLE 20
Calculated Mean Steady State PK Parameters

Pharmacokinetic Paramenters

| | | |
|---|---|---|
| Strength | Mean | Mean |

| mcg | mg | Cₘₐₓ (ng) | AUC₀₋₂₄ (ng · hr/mL) |
|---|---|---|---|
| 50 | 0.05 | 4.4 | 56.5 |
| 100 | 0.1 | 8.8 | 112.9 |
| 200 | 0.2 | 17.5 | 225.8 |
| 250 | 0.25 | 21.9 | 282.3 |
| 300 | 0.3 | 26.3 | 338.7 |
| 400 | 0.4 | 35.1 | 451.6 |
| 500 | 0.5 | 43.8 | 564.6 |
| 600 | 0.6 | 52.6 | 677.5 |
| 700 | 0.7 | 61.4 | 790.4 |
| 800 | 0.8 | 70.2 | 903.3 |
| 900 | 0.9 | 78.9 | 1016.2 |
| 1000 | 1 | 87.7 | 1129.1 |
| 1100 | 1.1 | 96.5 | 1242.0 |
| 1200 | 1.2 | 105.2 | 1354.9 |
| 1300 | 1.3 | 114.0 | 1467.8 |
| 1400 | 1.4 | 122.8 | 1580.7 |
| 1500 | 1.5 | 131.5 | 1693.7 |
| 2000 | 2.0 | 175.4 | 2258.2 |
| 2500 | 2.5 | 219.2 | 2822.8 |
| 3000 | 3.0 | 263.1 | 3387.3 |

The mean Tₘₐₓ value is calculated to be about 1.6 hours and mean T_{1/2} value is calculated to be about 19.4 hours.

### Example 10

Oglemilast tablet formulations ranging from 50 mcg to 3000 mcg are shown in Table 21. These formulations may be prepared using the procedure described in Example 8.

TABLE 21
Tablet Compositions (0.05 mg to 3 mg) prepared using Different Granules
and Tablet Weights

| Strength | | | | | | |
|---|---|---|---|---|---|---|
| 0.05 mg | 0.1 mg | 0.25 mg | 0.3 mg | 0.4 mg | 0.5 mg | 0.8 mg |
| 50 mcg | 100 mcg | 250 mcg | 300 mcg | 400 mcg | 500 mcg | 800 mcg |

| | |
|---|---|
| Ingredient | weight (grams per batch) |

Blend

| Oglemilast Sodium | 50 | | 50 | | 250 | | 150 | | 200 | | | | 400 | |
|---|---|---|---|---|---|---|---|---|---|---|---|---|---|---|
| Granules, 20 mg/g | | | | | | | | | | | | | | |
| Oglemilast Sodium | — | | — | | — | | | | | | 50 | | — | |
| Granules, 100 mg/g | | | | | | | | | | | | | | |
| Colloidal Silicon | 11 | | 11 | | 11 | | 11 | | 11 | | 5.5 | | 11 | |
| Dioxide | | | | | | | | | | | | | | |
| Sodium Starch | 50 | | 50 | | 50 | | 50 | | 50 | | 25 | | 50 | |
| Glycolate | | | | | | | | | | | | | | |
| Microcrystalline | 954 | | 954 | | 754 | | 894 | | 804 | | 452 | | 604 | |
| Cellulose/Colloidal | | | | | | | | | | | | | | |
| Silicon Dioxide | | | | | | | | | | | | | | |
| (ProSolv) | | | | | | | | | | | | | | |
| Talc | 30 | | 30 | | 30 | | 30 | | 30 | | 15 | | 30 | |
| Magnesium Stearate | 5 | | 5 | | 5 | | 5 | | 5 | | 2.5 | | 5 | |
| Total weight Final | 1100 | g | 1100 | g | 1100 | g | 1100 | g | 1100 | g | 550 | g | 1100 | g |
| Blend, grams | | | | | | | | | | | | | | |

Compression

| | | | | | | | | | | | | | | |
|---|---|---|---|---|---|---|---|---|---|---|---|---|---|---|
| Tablets Weight mg | 55 | mg | 110 | mg | 55 | mg | 110 | mg | 110 | mg | 55 | mg | 110 | mg |

| | Strength | | | | | | | | | | | |
|---|---|---|---|---|---|---|---|---|---|---|---|---|
| | 1.0 mg | | 1.2 mg | | 1.2 mg | | 1.5 mg | | 1.5 mg | | 3.0 mg | |
| | 1000 mcg | | 1200 mcg | | 1200 mcg | | 1500 mcg | | 1500 mcg | | 3000 mcg | |
| Ingredient | weight (grams per batch) | | | | | | | | | | | |
| Blend | | | | | | | | | | | | |
| Oglemilast Sodium | — | | — | | | | | | | | | |
| Granules, 20 mg/g | | | | | | | | | | | | |
| Oglemilast Sodium | 100 | | 120 | | 60 | | 150 | | 150 | | 75 | |
| Granules, 100 mg/g | | | | | | | | | | | | |
| Colloidal Silicon | 11 | | 11 | | 5.5 | | 11 | | 11 | | 5.5 | |
| Dioxide | | | | | | | | | | | | |
| Sodium Starch | 50 | | 50 | | 25 | | 50 | | 50 | | 25 | |
| Glycolate | | | | | | | | | | | | |
| Microcrystalline | 904 | | 884 | | 442 | | 854 | | 1554 | | 427 | |
| Cellulose/Colloidal | | | | | | | | | | | | |
| Silicon Dioxide | | | | | | | | | | | | |
| (ProSolv) | | | | | | | | | | | | |
| Talc | 30 | | 30 | | 15 | | 30 | | 30 | | 15 | |
| Magnesium Stearate | 5 | | 5 | | 2.5 | | 5 | | 5 | | 2.5 | |
| Total weight Final | 1100 | g | 1100 | g | 550 | g | 1100 | g | 1800 | g | 550 | g |
| Blend, grams | | | | | | | | | | | | |
| Compression | | | | | | | | | | | | |
| Tablets Weight mg | 110 | mg | 110 | mg | 220 | mg | 110 | mg | 180 | mg | 220 | mg |

### Example 11

Oglemilast tablet formulations prepared using different granulations are shown in Table 22. These formulations may be prepared using procedure described in Example 8.

TABLE 22
Examples of 0.8 mg and 0.9 Tablets Prepared Using Different Granules
and Tablet Weights

| Strength | | | |
|---|---|---|---|
| 0.9 mg | 0.9 mg | 0.8 mg | 0.8 mg |

| | |
|---|---|
| Ingredient | weight (gram per batch) |

Blend

| Oglemilast Sodium | 247.5 | | — | | — | | — | |
|---|---|---|---|---|---|---|---|---|
| Granules, 20 mg/g | | | | | | | | |
| Oglemilast Sodium | — | | 150 | | — | | — | |
| Granules, 40 mg/g | | | | | | | | |
| Oglemilast Sodium | — | | — | | 100 | | — | |
| Granules, 80 mg/g | | | | | | | | |
| Oglemilast Sodium | — | | — | | — | | 80 | |
| Granules, 100 mg/g | | | | | | | | |
| Colloidal Silicon Dioxide | 11 | | 11 | | 11 | | 11 | |
| Sodium Starch Glycolate | 50 | | 50 | | 50 | | 50 | |
| Microcrystalline | 804 | | 839 | | 904 | | 924 | |
| Cellulose/Colloidal | | | | | | | | |
| Silicon Dioxide | | | | | | | | |
| (ProSolv) | | | | | | | | |
| Talc | 30 | | 30 | | 30 | | 30 | |
| Magnesium Stearate, | 5 | | 5 | | 5 | | 5 | |
| Total weight of Final | 1100 | g | 1100 | g | 1100 | g | 1100 | g |
| Blend (grams) | | | | | | | | |

Compression

| | | | | | | | | |
|---|---|---|---|---|---|---|---|---|
| Tablets weight (mg) | 200 | mg | 165 | mg | 110 | mg | 110 | mg |

While the invention has been depicted and described by reference to exemplary embodiments of the invention, such a reference does not imply a limitation on the invention, and no such limitation is to be inferred. The invention is capable of considerable modification, alteration, and equivalents in form and function, as will occur to those ordinarily skilled in the pertinent arts having the benefit of this disclosure. The depicted and described embodiments of the invention are exemplary only, and are not exhaustive of the scope of the invention. Consequently, the invention is intended to be limited only by the spirit and scope of the appended claims, giving full cognizance to equivalence in all respects.

All references cited herein are hereby incorporated by reference in their entirety, except where stated otherwise.

1. A formulation comprising from about 0.05 to about 2.5 mg oglemilast or a pharmaceutically acceptable salt thereof, wherein the formulation provides an in vivo plasma profile comprising:
(i) a mean Cₘₐₓ of more than about 2 ng/mL,
(ii) a mean AUC₀₋₂₄ of more than about 26 ng.hr/mL and
(iii) a mean Tₘₐₓ of about 0.25 or more hours.

2. The formulation of claim 1, wherein the formulation comprises about 0.05 mg oglemilast or a pharmaceutically acceptable salt thereof.

3. The formulation of claim 1, wherein the formulation comprises about 0.1 mg oglemilast or a pharmaceutically acceptable salt thereof and provides an in vivo plasma profile comprising:
(i) a mean Cₘₐₓ of more than about 6 ng/mL,
(ii) a mean AUC₀₋₂₄ of more than about 100 ng.hr/mL and
(iii) a mean Tₘₐₓ of about 1 or more hours.

4. The formulation of claim 1, wherein the formulation comprises about 0.2 mg oglemilast or a pharmaceutically acceptable salt thereof, and provides an in vivo plasma profile comprising:
(i) a mean Cₘₐₓ of more than about 12 ng/mL,
(ii) a mean AUC₀₋₂₄ of more than about 150 ng.hr/mL and
(iii) a mean Tₘₐₓ of about 1 or more hours.

5. The formulation of claim 1, wherein the formulation comprises about 0.4 mg oglemilast or a pharmaceutically acceptable salt thereof, and provides an in vivo plasma profile comprising:
(i) a mean Cₘₐₓ of more than about 25 ng/mL,
(ii) a mean AUC₀₋₂₄ of more than about 240 ng.hr/mL and
(iii) a mean Tₘₐₓ of about 1 or more hours.

6. The formulation of claim 1, wherein the formulation comprises about 0.6 mg oglemilast or a pharmaceutically acceptable salt thereof, and provides an in vivo plasma profile comprising:
(i) a mean Cₘₐₓ of more than about 40 ng/mL,
(ii) a mean AUC₀₋₂₄ of more than about 550 ng.hr/mL and
(iii) a mean Tₘₐₓ of about 1 or more hours.

7. The formulation of claim 1, wherein the formulation comprises about 0.8 mg oglemilast or a pharmaceutically acceptable salt thereof, and provides an in vivo plasma profile comprising:
(i) a mean Cₘₐₓ of more than about 50 ng/mL,
(ii) a mean AUC₀₋₂₄ of more than about 650 ng.hr/mL and
(iii) a mean Tₘₐₓ of about 1 or more hours.

8. The formulation of claim 1, wherein the formulation comprises about 1.25 mg oglemilast or a pharmaceutically acceptable salt thereof, and provides an in vivo plasma profile comprising:
(i) a mean Cₘₐₓ of more than about 95 ng/mL,
(ii) a mean AUC₀₋₂₄ of more than about 1200 ng.hr/mL and
(iii) a mean Tₘₐₓ of about 1 or more hours.

9. The formulation of claim 1, wherein the formulation comprises about 2.5 mg oglemilast or a pharmaceutically acceptable salt thereof, and provides an in vivo plasma profile comprising:
(i) a mean Cₘₐₓ of more than about 150 ng/mL,
(ii) a mean AUC₀₋₂₄ of more than about 2400 ng.hr/mL and
(iii) a mean Tₘₐₓ of about 1 or more hours.

10. The formulation of claim 1, wherein the formulation comprises about 0.5 mg oglemilast or a pharmaceutically acceptable salt thereof, and provides an in vivo plasma profile comprising:
(i) a mean Cₘₐₓ of more than about 4 ng/mL,
(ii) a mean AUC₀₋₂₄ of more than about 30 ng.hr/mL and
(iii) a mean Tₘₐₓ of about 1 or more hours.

11. The formulation of claim 1, wherein the formulation comprises about 0.1 mg oglemilast or a pharmaceutically acceptable salt thereof, and provides an in vivo plasma profile comprising:
(i) a mean Cₘₐₓ of more than about 7 ng/mL,
(ii) a mean AUC₀₋₂₄ of more than about 120 ng.hr/mL and
(iii) a mean Tₘₐₓ of about 1 or more hours.

12. The formulation of claim 1, wherein the formulation comprises about 0.2 mg oglemilast or a pharmaceutically acceptable salt thereof, and provides an in vivo plasma profile comprising:
(i) a mean Cₘₐₓ of more than about 14 ng/mL,
(ii) a mean AUC₀₋₂₄ of more than about 140 ng.hr/mL and
(iii) a mean Tₘₐₓ of about 1 or more hours.

13. The formulation of claim 1, wherein the formulation comprises about 0.4 mg oglemilast or a pharmaceutically acceptable salt thereof, and provides an in vivo plasma profile comprising:
(i) a mean Cₘₐₓ of more than about 28 ng/mL,
(ii) a mean AUC₀₋₂₄ of more than about 260 ng.hr/mL and
(iii) a mean Tₘₐₓ of about 1 or more hours.

14. The formulation of claim 1, wherein the formulation comprises about 0.6 mg oglemilast or a pharmaceutically acceptable salt thereof, and provides an in vivo plasma profile comprising:
(i) a mean Cₘₐₓ of more than about 45 ng/mL,
(ii) a mean AUC₀₋₂₄ of more than about 600 ng.hr/mL and
(iii) a mean Tₘₐₓ of about 1 or more hours.

15. The formulation of claim 1, wherein the formulation comprises about 0.8 mg oglemilast or a pharmaceutically acceptable salt thereof, and provides an in vivo plasma profile comprising:
((i) a mean Cₘₐₓ of more than about 50 ng/mL,
(ii) a mean AUC₀₋₂₄ of more than about 600 ng.hr/mL and
(iii) a mean Tₘₐₓ of about 1 or more hours.

16. The formulation according to claim 1, wherein about 20% or more of the oglemilast is amorphous.

17. The formulation according to claim 1, wherein about 40% or more of the oglemilast is amorphous.

18. The formulation according to claim 1, wherein about 60% or more of the oglemilast is amorphous.

19. The formulation according to claim 1, wherein about 80% or more of the oglemilast is amorphous.

20. The formulation according to claim 1, wherein about 90% or more of the oglemilast is amorphous.

21. A method of treating an inflammatory or allergic condition comprising administering to a patient in need thereof a formulation according to claim 1.

22. The method of claim 21, wherein the condition is asthma, bronchial asthma, chronic obstructive pulmonary disease, allergic rhinitis, eosinophilic granuloma, nephritis, rheumatoid arthritis, cystic fibrosis, chronic bronchitis, multiple sclerosis, Crohns disease, psoraisis, uticaria, adult vernal cojunctivitis, respiratory distress syndrome, rhematoid spondylitis, osteoarthritis, gouty arthritis, uteltis, allergic conjunctivitis, inflammatory bowel conditions, ulcerative colitis, eczema, atopic dermatitis or chronic inflammation.

23. The method of claim 22, wherein the condition is asthma.

24. The method of claim 22, wherein the condition is COPD.

25. The method of claim 22, wherein the condition is rheumatoid arthritis.

26. A substantially pure amorphous form of a compound of formula (I): 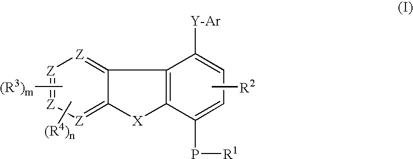
wherein
R¹, R² and R³ are each independently selected from the group consisting of hydrogen, substituted or unsubstituted alkyl, substituted or unsubstituted alkenyl, substituted or unsubstituted alkynyl, substituted or unsubstituted cycloalkyl, substituted or unsubstituted cycloalkylakyl, substituted or unsubstituted cycloalkenyl, substituted or unsubstituted aryl, substituted or unsubstituted arylalkyl, substituted or unsubstituted heteroaryl, substituted or unsubstituted heterocyclic group, substituted or unsubstituted heterocyclylalkyl, substituted or unsubstituted heteroarylalkyl, nitro, —OH, cyano, formyl, acetyl, halogen, protecting groups, —C(O)R^{a}, —C(O)OR^{a}, —C(O)NR^{a}R^{a}, —S(O)_{q}R^{a}, S(O)_{q}NR^{a}R^{a}, —NRR^{a}, —OR^{a}, and —SR^{a},
or two R³ substituents ortho to each other may be joined to a form a saturated or unsaturated 3-7 membered cyclic ring which may optionally include up to two heteroatoms which may be same or different selected from O, NR^{a} and S;
R⁴ is —NR⁵R⁶; wherein R⁵ and R⁶ are each independently selected from the group consisting of hydrogen, substituted or unsubstituted alkyl, substituted or unsubstituted alkenyl, substituted or unsubstituted alkynyl, substituted or unsubstituted cycloalkyl, substituted or unsubstituted cycloalkylakyl, substituted or unsubstituted cycloalkenyl, substituted or unsubstituted aryl, substituted or unsubstituted arylalkyl, substituted or unsubstituted heteroaryl, substituted or unsubstituted heterocyclic ring, substituted or unsubstituted heterocyclylalkyl, substituted or unsubstituted heteroarylalkyl, nitro, —OH, cyano, halogen, —C(O)R^{a}, —C(O)OR^{a}, —C(O)NRaR^{a}, —S(O)_{q}R^{a}, —S(O)_{q}NR^{a}R^{a}, —C(═NR^{a})R^{a}, —C(═NR^{a})NR^{a}R^{a}, —C(═S)NR^{a}R^{a}, —C(═S)R^{a}, —N═C(R^{a}R^{a})—, —NR^{a}R^{a}, —OR^{a}, —SR^{a}, and protecting groups,
or R⁵ and R⁶ may be joined to a form a saturated or unsaturated 3-7 membered cyclic ring, which may optionally include up to two heteroatoms which may be same or different selected from O, NR^{a} and S;
Ar is selected from the group consisting of substituted or unsubstituted aryl, substituted or unsubstituted arylalkyl, substituted or unsubstituted heterocyclic ring and substituted or unsubstituted heteroaryl ring;
X is selected from the group consisting of O, S(O)_{q} and NR^{a};
Y is selected from the group consisting of —C(O)NR⁷, —NR⁷S(O)_{q}, —S(O)_{q}NR⁷ and —NR⁷C(O);
each Z is independently C or N;
R⁷ is selected from the group consisting of hydrogen, substituted or unsubstituted alkyl, hydroxyl, —OR^{a}, substituted or unsubstituted aryl, and substituted or unsubstituted heterocyclic ring;
p is chosen from O and S;
m represents 0-3; n represents 1-4; q represents 0, 1 or 2; and
R^{a} is selected from the group consisting of hydrogen, substituted or unsubstituted alkyl, substituted or unsubstituted alkenyl, substituted or unsubstituted alkynyl, substituted or unsubstituted cycloalkyl, substituted or unsubstituted cycloalkylakyl, substituted or unsubstituted cycloalkenyl, substituted or unsubstituted aryl, substituted or unsubstituted arylalkyl, substituted or unsubstituted heteroaryl, substituted or unsubstituted heterocyclic ring, substituted or unsubstituted heterocyclylalkyl, substituted or unsubstituted heteroarylalkyl, nitro, —OH, cyano, formyl, acetyl, halogen, protecting groups, —C(O)Ra, —C(O)OR^{a}, —C(O)NR^{a}R^{a}, —S(⁰)_{q}R^{a}, —S(O)_{q}NR^{a}R^{a}, —N^{a}R^{a}, —OR^{a}, and —SR^{a}.

27. The compound of claim 26, wherein about 40% or more of the compound is amorphous.

28. The compound of claim 26, wherein about 60% or more of the compound is amorphous

29. The compound of claim 26, wherein about 75% or more of the compound is amorphous

30. The compound of claim 26, wherein about 80% or more of the compound is amorphous

31. The compound of claim 26, wherein about 90% or more of the compound is amorphous.

32. The compound of claim 26, wherein the compound comprises oglemilast.

33. A formulation comprising about 20% or more of an amorphous compound of formula (I): 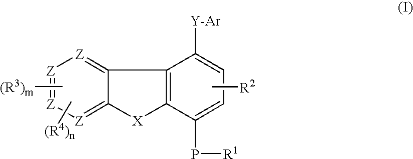
wherein
R¹, R² and R³ are each independently selected from the group consisting of hydrogen, substituted or unsubstituted alkyl, substituted or unsubstituted alkenyl, substituted or unsubstituted alkynyl, substituted or unsubstituted cycloalkyl, substituted or unsubstituted cycloalkylakyl, substituted or unsubstituted cycloalkenyl, substituted or unsubstituted aryl, substituted or unsubstituted arylalkyl, substituted or unsubstituted heteroaryl, substituted or unsubstituted heterocyclic group, substituted or unsubstituted heterocyclylalkyl, substituted or unsubstituted heteroarylalkyl, nitro, —OH, cyano, formyl, acetyl, halogen, protecting groups, —C(O)R^{a}, —C(O)OR^{a}, —C(O)NR^{a}R^{a}, —S(O)_{q}R^{a}, S(O)_{q}NR^{a}R^{a}, —NRR^{a}, —OR^{a}, and —SR^{a},
or two R³ substituents ortho to each other may be joined to a form a saturated or unsaturated 3-7 membered cyclic ring which may optionally include up to two heteroatoms which may be same or different selected from O, NR^{a} and S;
R⁴ is —NR⁵R⁶; wherein R⁵ and R⁶ are each independently selected from the group consisting of hydrogen, substituted or unsubstituted alkyl, substituted or unsubstituted alkenyl, substituted or unsubstituted alkynyl, substituted or unsubstituted cycloalkyl, substituted or unsubstituted cycloalkylakyl, substituted or unsubstituted cycloalkenyl, substituted or unsubstituted aryl, substituted or unsubstituted arylalkyl, substituted or unsubstituted heteroaryl, substituted or unsubstituted heterocyclic ring, substituted or unsubstituted heterocyclylalkyl, substituted or unsubstituted heteroarylalkyl, nitro, —OH, cyano, halogen, —C(O)R^{a}, —C(O)OR^{a}, —C(O)NRaR^{a}, —S(O)_{q}R^{a}, —S(O)_{q}NR^{a}R^{a}, —C(═NR^{a})R^{a}, —C(═NR^{a})NR^{a}R^{a}, —C(═S)NR^{a}R^{a}, —C(═S)R^{a}, —N═C(R^{a}R^{a})—, —NR^{a}R^{a}, —OR^{a}, —SR^{a}, and protecting groups,
or R⁵ and R⁶ may be joined to a form a saturated or unsaturated 3-7 membered cyclic ring, which may optionally include up to two heteroatoms which may be same or different selected from O, NR^{a} and S;
Ar is selected from the group consisting of substituted or unsubstituted aryl, substituted or unsubstituted arylalkyl, substituted or unsubstituted heterocyclic ring and substituted or unsubstituted heteroaryl ring;
X is selected from the group consisting of O, S(O)_{q} and NR^{a};
Y is selected from the group consisting of —C(O)NR⁷, —NR⁷S(O)_{q}, —S(O)_{q}NR⁷ and —NR⁷C(O);
each Z is independently C or N;
R⁷ is selected from the group consisting of hydrogen, substituted or unsubstituted alkyl, hydroxyl, —OR^{a}, substituted or unsubstituted aryl, and substituted or unsubstituted heterocyclic ring;
p is chosen from O and S;
m represents 0-3; n represents 1-4; q represents 0, 1 or 2; and
R^{a} is selected from the group consisting of hydrogen, substituted or unsubstituted alkyl, substituted or unsubstituted alkenyl, substituted or unsubstituted alkynyl, substituted or unsubstituted cycloalkyl, substituted or unsubstituted cycloalkylakyl, substituted or unsubstituted cycloalkenyl, substituted or unsubstituted aryl, substituted or unsubstituted arylalkyl, substituted or unsubstituted heteroaryl, substituted or unsubstituted heterocyclic ring, substituted or unsubstituted heterocyclylalkyl, substituted or unsubstituted heteroarylalkyl, nitro, —OH, cyano, formyl, acetyl, halogen, protecting groups, —C(O)Ra, —C(O)OR^{a}, —C(O)NR^{a}R^{a}, —S(O)_{q}R^{a}, —S(O)_{q}NR^{a}R^{a}, —N^{a}R^{a}, —OR^{a}, and —SR^{a}.

34. The formulation of claim 33, wherein about 40% or more of the compound of formula (I) is amorphous.

35. The formulation of claim 33, wherein about 60% or more of the compound of formula (I) is amorphous.

36. The formulation of claim 33, wherein about 75% or more of the compound of formula (I) is amorphous.

37. The formulation of claim 33, wherein about 80% or more of the compound of formula (I) is amorphous.

38. The formulation according to claim 33, wherein about 90% or more of the compound of formula (I) is amorphous.

39. The formulation of claim 33, wherein the formulation comprises oglemilast.

40. The formulation of claim 39, further comprising oglemilast sodium.

41. A formulation comprising about 20% or more amorphous oglemilast, wherein the formulation provides an in vivo plasma profile comprising:
(i) a mean Cₘₐₓ of more than about 2 ng/mL,
(ii) a mean AUC_{0-∞} of less than about 15,000 ng h/ml, and
(iii) a mean Tₘₐₓ of more than about 0.25 hour.

42. The formulation of claim 41, wherein about 40% or more of the oglemilast is amorphous.

43. The formulation of claim 41, wherein about 60% or more of the oglemilast is amorphous.

44. The formulation of claim 41, wherein about 80% or more of the oglemilast is amorphous.

45. The formulation of claim 41, wherein about 90% or more of the oglemilast is amorphous.

46. The formulation of claim 41, comprising from about 0.05 to about 2.5 mg oglemilast.

47. The formulation of claim 41, comprising from about 0.1 to about 2.5 mg oglemilast.

48. The formulation of claim 41, comprising from about 0.2 to about 1 mg oglemilast.

49. A formulation comprising 0.8 mg oglemilast wherein about 20% or more of the oglemilast is amorphous and the formulation provides an in vivo plasma profile comprising:
(i) a mean Cₘₐₓ of more than about 38 ng/mL,
(ii) a mean AUC_{0-∞}0 of more than about 440 ng h/ml, and
(iii) a mean Tₘₐₓ of more than about 0.25 hours.

50. (canceled)

51. The formulation of claim 49, wherein the formulation has a dissolution rate of the active ingredient of about 85% or more in about 60 minutes or less.

52. The formulation of claim 49, wherein the formulation has a dissolution rate of the active ingredient of about 80% or more in about 60 minutes or less.

53. A formulation comprising (i) solubilized form of oglemilast and (ii) one or more excipients wherein the one or more excipients are present in an amount sufficient to retard formation of crystalline oglemilast.

54. The formulation of claim 53 in the form of a solution or a suspension.

55. The formulation of claim 54, wherein the pH of the solution is greater than about 7.

56. The formulation of claim 53, where the one or more excipients are selected from povidone, polyethylene glycol, celluloses, starches, povidone-vinyl acetate copolymers, cyclodextrins, disaccharides, polysaccharides and combinations thereof.

57. The formulation of claim 56, where the one or more excipients are selected from povidone, polyethylene glycol, hydroxypropyl methylcellulose, hydroxypropyl cellulose, pregelatinized starch, povidone-vinyl acetate copolymers hydroxypropyl beta cyclodextrin, sucrose, trehalose, dextran, and combinations thereof.

58. The formulation of claim 57, wherein the excipient is povidone.

59. The formulation of claim 33, wherein the formulation is adapted for oral administration.

60. The formulation of claim 59, in the form of a pill, tablet, capsule, dragee, powder, caplet, troche, elixir, oral suspension, solution, dry powder suspension, syrup, wafer, lozenge, orally disintegrating film, or orally disintegrating tablet.

61. The formulation of claim 60, in the form of a tablet, capsule, solution or oral suspension.

62. A method of treating an inflammatory or allergic condition comprising administering to a patient in need thereof a formulation according to claim 33.

63. The method of claim 62, wherein the inflammatory or allergic condition is chosen from asthma, bronchial asthma, chronic obstructive pulmonary disease, allergic rhinitis, eosinophilic granuloma, nephritis, rheumatoid arthritis, cystic fibrosis, chronic bronchitis, multiple sclerosis, Crohns disease, psoraisis, uticaria, adult vernal cojunctivitis, respiratory distress syndrome, rhematoid spondylitis, osteoarthritis, gouty arthritis, uteltis, allergic conjunctivitis, inflammatory bowel conditions, ulcerative colitis, eczema, atopic dermatitis and chronic inflammation.

64. The method of claim 63, wherein the condition is asthma.

65. The method of claim 63, wherein the inflammatory or allergic condition is COPD.

66. The method of claim 63, wherein the inflammatory condition is rheumatoid arthritis.

67. A formulation comprising about 20% solubilized oglemilast, wherein the formulation provides an in vivo plasma profile comprising:
(i) a mean Cₘₐₓ of more than about 2 ng/mL,
(ii) a mean AUC_{0-∞} of less than about 15,000 ng h/ml, and
(iii) a mean Tₘₐₓ of more than about 0.25 hour.
